(19) United States
(12) Patent Application Publication
(10) Pub. No.: US 2010/0174212 A1
(43) Pub. Date: Jul. 8, 2010

(54) Sexual quotient systems and methods

(76) Inventor: Youngtack Shim, Port Moody (CA)

Correspondence Address: Youngtack Shim, 155 Aspenwood Drive, Port Moody, BC, V3H 5A5, CA

(21) Appl. No.: 12/318,660

(22) Filed: Jan. 6, 2009

(51) Int. Cl. A61B 5/103; A61F 5/00

(52) U.S. Cl. 600/587; 600/38

(57) ABSTRACT

The present invention generally relates to systems for assessing quantitative quotients related to various static and dynamic characteristics of pelvic (i.e., vaginal) structures of females and penile structures of males associated with sexual activities. More particularly, the present invention relates to various systems capable of measuring one or multiple variables of such structures each of which represents a physiologic state of such structures, obtaining values of such variables, and assessing various sexual quotients such as vaginal quotients for females and penile quotients for males. Such a system may include at least one sensor unit for measuring such variables and at least one control unit for processing sensing signals generated by the sensor unit and assessing such quotients according to a preset format. The present invention also relates to various methods of assessing such quotients from the values of various variables and matching a given vaginal quotient with one of multiple penile quotients (or vice versa). The present invention also relates to various processes for making various members, units, and/or parts of the above system and providing various formats of such quotients.

### CROSS-REFERENCES TO RELATED APPLICATIONS

The present application claims an earlier invention date of the Disclosure Document entitled the same, deposited in the U.S. Patent and Trademark Office (the “Office”) on Jan. 12, 2007 under the Disclosure Document Deposit Program of the Office, and bearing the Serial Number 611,025 an entire portion of which is incorporated herein by reference.

### FIELD OF THE INVENTION

The present invention generally relates to systems for assessing quantitative quotients related to various static and dynamic characteristics of pelvic (i.e., vaginal) structures of females and penile structures of males associated with sexual activities. More particularly, the present invention relates to various systems capable of measuring one or multiple variables of such structures each of which represents a physiologic state of such structures, obtaining values of such variables, and assessing various sexual quotients such as vaginal quotients for females and penile quotients for males. Such a system may include at least one sensor unit for measuring such variables and at least one control unit for processing sensing signals generated by the sensor unit and assessing such quotients according to a preset format. The present invention also relates to various methods of assessing such quotients from the values of various variables and matching a given vaginal quotient with one of multiple penile quotients (or vice versa). The present invention also relates to various processes for making various members, units, and/or parts of the above system and providing various formats of such quotients.

### BACKGROUND OF THE INVENTION

A vaginal anatomy typically includes a vaginal entry and a vaginal wall, where such an entry defines an orifice therethrough, while the wall includes muscles and defines a vaginal cavity which extends inwardly from the entry and which is also bound by such muscles. The entry forms a clitoris thereon, and a paraurethral gland of an urethral sponge of a clitoris (also called the Grafenberg spot or G spot) is believed to be defined on the wall. The vaginal wall is formed essentially of two sets of muscles, the former extending longitudinally while the latter encircling the vagina. These muscles are specifically termed as “pubococygenus” and “levator ani” and are are located immediately adjacent to the vagina. These muscles have general appearance of a hammock with its two ends connected to the sides of a pelvis. In particular, the pubococygenus is basically a sphincter muscle which passes through a middle third of the vagina and runs in a circular band, with a ring-like ridges forming a part of a urethra and anus. For simplicity of illustration, this vaginal anatomy is to be referred to as “a pelvic structure,” the vaginal entry as an “entry,” and the vaginal wall as a “wall.” In addition, such a pelvic structure is referred to as the “standard pelvic structure” or simply the “pelvic structure” hereinafter for simplicity of illustration, unless otherwise specified.

Among devices currently available in the market for enhancing sexual functioning are dildos, vaginal exercise bars, and prostate stimulators. These devices generally provide stimuli from friction upon manipulation of their stimulators or by pressure due to distention of the pelvic cavity effected by a volume of such stimulators.

For example, manual pelvic relaxing devices have been proposed in various configurations as disclosed in various prior art such as, e.g., U.S. Pat. No. 3,996,930 to Sekulich, U.S. Pat. No. 5,690,603 to Kain, U.S. Pat. No. 5,690,604 to Barnett, U.S. Pat. No. 5,853,362 to Jacobs, U.S. Pat. No. 6,203,491 to Uribe, U.S. Pat. App. Pub. No. 2005/0187431 by Hudson, U.S. Pat. App. Pub. No. 2005/0228218 by Skidmore et al., U.S. Pat. No. 6,540,667 to Hickman, and the like. Being manual, users have to manually move such devices in and out of the pelvic cavity and/or around the pelvic opening.

In order to overcome inconvenience thereof, various automatic mechanisms have been added to such pelvic relaxing devices. In one class of examples, electric motors or electromagnetic vibration mechanisms have been incorporated to various automatic devices for effecting vibration as disclosed in various prior art such as, e.g., U.S. Pat. No. 3,451,391 to Tavel, U.S. Pat. No. 3,504,665 to Bakunin et al., U.S. Pat. No. 3,626,931 to Bysakh, U.S. Pat. No. 3,669,100 to Csanad, U.S. Pat. No. 3,991,751 to O'Rourke, U.S. Pat. No. 4,788,968 to Rudashevsky et al., U.S. Pat. No. 5,067,480 to Woog et al, U.S. Pat. No. 6,056,705 to Stigar-Brown, U.S. Pat. Appl. Pub. 2004/0034315 to Chen, U.S. Pat. Appl. Pub. 2004/0127766 to Chen, and the like.

In another class of example, automatic pelvic relaxing devices have used various mechanisms of converting rotational movements generated by such electric motors into translational movements for effecting horizontal and/or vertical translation of their stimulators. Several examples of such prior art include U.S. Pat. No. 4,722,327 to Harvey, U.S. Pat. No. 4,790,296 to Segal, U.S. Pat. No. 5,076,261 to Black, U.S. Pat. No. 5,725,473 to Taylor, U.S. Pat. No. 6,142,929 to Padgett, U.S. Pat. No. 6,422,993 to Hudson, U.S. Pat. No. 6,866,645 to Lee, and U.S. Pat. Appl. Pub. 2004/0147858

Various automatic pelvic relaxing devices have also used various mechanisms for converting rotational movements generated by such electric motors into lateral movements for effecting horizontal translation of their stimulators along a direction generally normal to axes of such stimulators. Several examples of such prior art are U.S. Pat. No. 5,460,597 to Hopper, U.S. Pat. No. 5,470,303 to Leonard et al., and U.S. Pat. No. 5,851,175 to Nickell.

Other therapeutic devices, although developed for various purposes other than pelvic relaxing, seem to have been used as alternatives as such conventional pelvic relaxing devices. In one class of examples, various manual or vibration devices have been disclosed to train or heal pelvic muscles as exemplified in U.S. Pat. No. 3,598,106 to Buning, U.S. Pat. No. 4,241,912 to Mercer et al., and U.S. Pat. No. 4,574,791 to Mitchener. In another class of examples, various devices have been developed for massaging various portions of a human body as disclosed in U.S. Pat. No. 4,055,170 to Nohmura, U.S. Pat. No. 4,825,853 to Iwamoto et al., U.S. Pat. No. 4,846,158 to Teranishi, U.S. Pat. No. 4,911,149 to Borodulin et al., U.S. Pat. No. 5,063,911 to Teranishi, and the like. In another class, massage devices have also been devised to provide translational movements as disclosed in U.S. Pat. No. 4,002,164 to Bradley, U.S. Pat. No. 5,085,207 to Fiore, U.S. Pat. No. 5,676,637 to Lee, and the like. In another class of examples, various devices have also been arranged to provide rotating, tapping, swinging and/or swiveling movements as described in U.S. Pat. No. 4,162,675 to Kawada, U.S. Pat. No. 6,632,185 to Chen, U.S. Pat. No. 4,088,128 to Mabuchi, U.S. Pat. No. 4,513,737 to Mabuchi, U.S. Pat. No. 4,827,914 to Kamazawa, U.S. Pat. No. 4,834,075 to Guo et al., U.S. Pat. No. 5,183,034 to Yamasaki et al., and U.S. Pat. No. 6,402,710 to Hsu. A vacuum device of U.S. Pat. No. 4,033,338 to lgwebike as well as a balloon device of U.S. Pat. No. 4,050,449 to Castellana et al. have also been proposed.

Regardless of their detailed mechanisms and/or movements effected thereby, all of these prior art devices suffer from common drawbacks. Excluding those manual ones, typical automatic devices consist of main modules and control modules which operatively couple with the main modules by wire for delivering electric power and control signals. Such wire, however, tends to be easily tangled and damaged. To overcome this defect, modern automatic pelvic relaxing device are fabricated as single unitary articles each with a main body and a handle which fixedly couples with a top part of the main body. The main body is generally designed to be inserted into the pelvic cavity, whereas the handle is shaped and sized to provide a grip for the user and also incorporates therein various control buttons. Accordingly, the handle consists of a space to form the grip and another space for such buttons. In order to avoid providing an inadvertently long device, however, a part of the handle closer to the main body is recruited to define the grip, whereas the rest of the handle houses the control buttons. It is to be appreciated, however, that all control buttons of conventional automatic devices are either on/off switches or speed control switches, where the on/off switches turn on and off the entire device or a specific movement thereof, and the speed control switches control a speed of the specific movement. In addition, such switches are typically designed to be activated and deactivated each time the user presses or touches them. Accordingly, when the user inadvertently touches any of such on/off and control switches during use, the device may be accidentally turned off, change speeds, and the like. In order to avoid such inadvertent operation, the control buttons have been incorporated as far away from the grip space of the handle, which in turn causes the very inconvenience of requiring the user to change the grip or to move his or her hand to manipulate the control buttons during operation when the user wants to change the speed of movement.

In contrary to these devices, novel pelvic relaxing systems, methods, and/or processes have already been conceived of and disclosed in numerous co-pending Applications of the same Applicant. For example, various pelvic relaxing systems have been proposed for manipulating their input and/or sensor units without mandating the user to change the grip, for providing various stimuli to the clitoris and/or G-spot of the user, for providing interactive capabilities thereto, for synchronizing movements of their various parts and/or operations thereof with internal and/or external signals, for incorporating electric stimulators thereinto, for installing the body members capable of adjusting their configurations, for incorporating retention mechanisms thereinto, for providing feedback mechanisms thereto, and for generating reciprocating movements of only portions of body members thereof. Although these novel systems solve most deficiencies of the conventional devices, none of them are capable of assisting the user in improving muscle tones of her pelvic structure.

It is well recognized in the field that improved muscle strengths and tones of various muscles of the pelvic structure are advantageous in many respects. For example, the stronger pelvic muscles are generally attributed to experiencing an orgasm or to having more intense and/or multiple orgasms during a sexual intercourse. To this end, various modalities have been proposed to improve tones of the pelvic muscles among which the most popular is the “Kegel” exercise. Similar to other exercising modalities, the Kegel exercise hinges on a well-accepted principle of “Specific Adaptation to Imposed Demand” which dictates that muscles will adapt only to demands placed thereupon. Accordingly, one need to place an ever-increasing demand on her muscles for continued improvement thereof, e.g., by adding an extra repetition to an exercise, increasing demand (or load) on the muscles, shortening rest intervals during the exercise, and the like. Accordingly, various conventional pelvic exercise devices urge the user to add progressive dynamic resistance to conventional Kegel exercise devices.

Although conventional Kegel exercise devices are generally effective in improving the muscle strengths and tones, such devices suffer from several common deficiencies. For example, almost all conventional devices use sensors filled with air, and assess physiologic states of various muscles by monitoring changes in pressure of air trapped inside the sensors. The sensor is generally disposed in an insertable unit of the device and arranged to be in fluid communication with a monitoring unit such as an analog pressure gauge or a digital display through a tubing which is typically made of polymers. Because air is a compressible gas, however, such air pressure inside the sensor generally depends upon temperature of a surrounding medium. Therefore, the air trapped in the sensor is heated to body temperature upon insertion of the insertable unit into the internal cavity of the structure, and expands due to an increase in temperature. Because the tubing is disposed in room temperature, however, a mismatch between such temperatures may cause measurement error in the air pressure. In addition, the compliant tubing between the sensor and monitoring unit may change its internal volume when the tubing is moved around. In addition, such a conventional device is typically forced to employ a single sensor, for it is not readily amenable to incorporate two compliant air-filled sensors. Therefore, such a device employs the sensor over at least a substantial part of its insertable unit and only provides a value of the air pressure averaged over the part of the sensor. Moreover, such conventional devices are directed to measure the pressure exerted by the pelvic muscles while the user maintains a fixed posture and cannot assess configurational changes in the pelvic structure as the user is in a different posture.

Such Kegel devices are by far only ones capable of quantitatively assessing the force and/or electrical potentials accompanying the contraction and relaxation of the pelvic muscles. It is manifest, however, that static and dynamic characteristics of the pelvic structure may not be quantified solely by the force and electric potential generated by such pelvic muscles. As a matter of fact, the internal cavity of the pelvic structure defines a tortuous configuration which extends along a curved direction and has varying diameters therealong. In addition, the pelvic muscles are not homogeneous, i.e., such pelvic muscles wrap the internal cavity in a helical fashion and in varying thicknesses. Therefore, the forces exerted by such muscles are not exerted along a single direction but in three dimensions, and magnitudes of such forces vary not only along the internal cavity but also around a periphery of such a cavity defined perpendicular to the curvilinear axis of the cavity. Moreover, such static and dynamic characteristics of the pelvic structure depend heavily upon a posture thereof, for changing a posture necessitates contraction, stretching, and/or repositioning of other muscles and/or bones. None of the prior art devices can account for such static and dynamic characteristics of the pelvic structure and, therefore, can provide any meaning indices characterizing such characteristics.

Accordingly, there is a need for a pelvic quotient system capable of measuring various pelvic variables and assessing therefrom quantitative quotients which represent static and dynamic states of the pelvic structure. There also is a need for a pelvic quotient system capable of assessing such quotients based upon a preset format and embodying such quotients into tangible articles. In addition, there is a need for another system capable of assessing quantitative quotients for penile structures of males and matching the female who defines a preset pelvic quotient with the male defining a matching quotient of the penile structure.

### SUMMARY OF THE INVENTION

The present invention generally relates to systems for assessing quantitative quotients related to various static and dynamic characteristics of pelvic (i.e., vaginal) structures of females and penile structures of males associated with sexual activities. More particularly, the present invention relates to various systems capable of measuring one or multiple variables of such structures each of which represents a physiologic state of such structures, obtaining values of such variables, and assessing various sexual quotients such as vaginal quotients for females and penile quotients for males. Such a system may include at least one sensor unit for measuring such variables and at least one control unit for processing sensing signals generated by the sensor unit and assessing such quotients according to a preset format. Such a system may have a single sensor unit but dispose such an unit in multiple portions of the pelvic structure for measuring multiple values of a single variable. Such a system may instead include multiple sensor units and measure different variables in a single or multiple portions of the pelvic structure. The control unit may assess the quotients in various formats such as, e.g., pure numbers, numbers and symbols (including alphabets), a set of numbers and/or symbols, and the like. The system may also assess such quotients in sounds, images, and tangible articles such as various emblems. The present invention also relates to another system capable of measuring corresponding variables of the penile structure and assessing corresponding quotients. The present invention also relates to various systems capable of matching such quotients of females with those of males based upon various formats.

The present invention relates to various methods of assessing such quotients from the values of various variables and matching a given vaginal quotient with one of multiple penile quotients (or vice versa). The present invention also relates to various methods of measuring the pelvic variables using a single or multiple sensor units, measuring such variables in a single or multiple portions of the pelvic structure, and assessing the quotients from the values of such variables. The present invention also relates to further methods of generating such quotients by a single number and/or symbol, by a set of multiple numbers and/or symbols, providing such quotients in sounds and/or images, embodying such quotients in the tangible articles, and the like. The present invention further relates to various methods of matching such quotients of the pelvic structures of females with those of the penile structures of males (or vice versa). The present invention also relates to various processes for producing various members, units, and/or parts of the above system and providing various formats of such quotients.

Therefore, one objective of the present invention is to provide a pelvic quotient system which may express various static and/or dynamic characteristics of physiologic states of a pelvic structure in terms of a quantitative quotient. Thus, a related objective of the present invention is to provide the system capable of assessing such a quotient in terms of values of various variables measured and/or assessed by such a system. Another related objective of this invention is to provide another system which may similarly express various static and/or dynamic characteristics of physiologic states of a penile structure in terms of another quantitative quotient, in particularly, in terms of values of various variables measured and/or assessed by the system.

Another objective of the present invention is to provide such a pelvic quotient system which may express the above vaginal (or pelvic) quotient in a single number or symbol, in multiple numbers and/or symbols, in a set of numbers and/or symbols, and the like, where each of such numbers and symbols may represent the static or dynamic characteristics of the pelvic structure or a configuration thereof. A related objective of this invention is to provide a system which may similarly express the above penile quotient in such numbers, symbols, and/or sets thereof. Another related objective of this invention is to provide a system capable of measuring one or multiple variables of such structures for assessing such quotients as described in this paragraph.

Another objective of the present invention is to provide such a pelvic quotient system which may express the above vaginal quotient in sounds. Therefore, a related objective of this invention is to provide a system capable of generating the sounds while manipulating amplitudes of such sounds, tones thereof, pitches thereof, melodies thereof, and/or content bases carried thereby to represent such a quotient. Another related objective of this invention is to provide a system which may similarly express the above penile quotient in sounds, while manipulating such characteristics of the sounds to express the penile quotient. Another related objective of the present invention is to provide a system capable of measuring one or more multiple variables of the structures for assessing such quotients as described in this paragraph.

Another objective of the present invention is to provide such a pelvic quotient system which may express the above vaginal quotient in images. Accordingly, a related objective of this invention is to provide a system capable of generating the images while manipulating shapes of the images, sizes thereof, colors thereof, and contents thereof to represent such a quotient. Another related objective of this invention is to provide a system which may similarly express the above penile quotient in such images. Another related objective of this invention is to provide a system capable of measuring one or multiple variables of such structures for assessing such quotients as described in this paragraph.

Another objective of the present invention is to provide such a pelvic quotient system which may express the above vaginal quotient in tangible articles such as emblems, locks, keys, and so on. Accordingly, a related objective of the present invention is to provide a system capable of generating the tangible articles while manipulating shapes of such articles, sizes thereof, surface characteristics including protrusions and/or indentations thereof, curvatures thereof, and colors thereof to represent such a quotient. Another related objective of this invention is to provide a system which may similarly express the penile quotient in such articles. Another related objective of this invention is to provide a system capable of measuring one or multiple variables of such structures for assessing the quotients as described in this paragraph. A yet another related objective of this invention is to provide a system capable of expressing such vaginal and/or penile quotients in tangible articles which express or mimic external and/or internal configurations of the pelvic and/or penile structures.

Another objective of the present invention is to provide such a pelvic quotient system which may assess multiple vaginal quotients of the structure as a subject maintains a preset posture of the structure. Therefore, a related objective of the present invention is to provide the system capable of assessing multiple vaginal quotients in one posture, identifying an optimum quotient, and notifying the quotient and posture to the subject. Another related objective of this invention is to provide a system which may similarly assess multiple penile quotients, identify the quotient beyond a preset threshold, and notify the subject of the quotient and posture.

Another objective of the present invention is to provide such a pelvic quotient system which may assess multiple vaginal quotients of the structure while the subject changes postures of such a structure. Therefore, a related objective of the present invention is to provide the system capable of assessing multiple vaginal quotients in multiple postures, identifying an optimum quotient, and notifying the quotient and corresponding posture to the subject. Another related objective of this invention is to provide a system which may similarly assess multiple penile quotients while the subject changes the postures, identify the quotient beyond a preset threshold, and notify the subject of the quotient as well as the posture.

Another objective of the present invention is to provide such a pelvic quotient system which may assess a series of different postures in which vaginal quotients of the structure always exceed a preset threshold. Accordingly, a related objective of the present invention is to provide the system capable of assessing multiple vaginal quotients in multiple postures, identifying those quotients beyond the threshold, and arranging multiple postures corresponding to those quotients into a feasible order in which the quotients always exceed the preset threshold. Another related objective of this invention is to provide a system which may similarly assess multiple penile quotients as the subject changes such postures, identify the quotients exceeding such a threshold, generate a sequence of such postures in each of which the quotients exceed the threshold, and notify the subject of the sequence or order of such postures.

Another objective of the present invention is to provide such a pelvic quotient system which may obtain the vaginal and penile quotients and assess a degree or extent of matching between such quotients. A related objective of this invention is to provide a system which may match the quotients primarily based upon signs of, magnitudes of, a similarity between, and/or a difference between such quotients. Another related objective of the present invention is to provide a system which may match such quotients primarily based upon a sum, a product, and/or a ratio of such quotients.

Another objective of the present invention is to provide such a pelvic quotient system which may obtain the vaginal and penile quotients as sets of numbers and/or symbols and assess a degree or an extent of matching between such quotients. A related objective of this invention is to provide a system which may match such quotients primarily comparing a sign of a number and/or symbol of the vaginal quotient with that of a corresponding number and/or symbol of the penile quotient, which may match such quotients primarily comparing a magnitude of a number of the vaginal quotient with that of a corresponding number of the penile quotient, and the like. Another related objective of this invention is to provide a system capable of matching such quotients primarily based upon a similarity between profiles of the quotients along a preset direction, a difference between such profiles of the quotients in such a direction, a complementarity between such profiles of the quotients along the direction, and the like.

Another objective of the present invention is to provide such a pelvic quotient system which may obtain the vaginal and penile quotients as audible (or audio) signals and then assess a degree or extent of matching between the quotients. A related objective of this invention is to provide a system capable of matching such quotients primarily based upon amplitudes of such signals, tomes thereof, pitches thereof, melodies thereof, and/or content bases carried thereby.

Another objective of the present invention is to provide such a pelvic quotient system which may obtain the vaginal and penile quotients as two- or three-dimensional shapes and assess a degree or extent of matching between such quotients. A related objective of this invention is to provide a system capable of matching such quotients primarily based upon sizes of the shapes, lengths thereof, heights thereof, widths thereof, thicknesses thereof, a similarity or congruence between the shapes, a difference therebetween, an angle defined between such shapes when they are overlapped onto each other, and the like. Another related objective of this invention is to provide a system capable of matching such quotients primarily based on a number of holes defined in such shapes, sizes thereof, locations thereof, a degree of overlapping between such holes when overlapped onto each other, an angle defined between such shapes when a preset number of such holes are overlapped onto each other, and the like. Another related objective of the present invention is to provide a system capable of matching such quotients primarily based on a number of protrusions and/or indentations defined in such shapes, sizes thereof, locations thereof, a degree of matching between at least one protrusion of one of such shapes and at least one indentation of the other thereof, an angle formed between the shapes when at least one of said protrusions and indentations of such shapes are aligned with each other.

Another objective of the present invention is to provide such a pelvic quotient system which may obtain the vaginal and penile quotients in monochromic and/or multichromic colors and assess a degree or extent of matching between such quotients. Therefore, a related objective of this invention is to provide a system capable of matching such quotients primarily based upon hues of such colors, brightness thereof, contrast therebetween, another color resulting from mixing at least two of such colors, another color obtained by overlapping such colors, and the like.

Another objective of the present invention is to provide such a pelvic quotient system which may obtain the vaginal and penile quotients in patterns defining one or more objects therein and then assess a degree or extent of matching between the quotients. A related objective of this invention is to provide a system capable of matching such quotients primarily based on shapes of such objects of the patterns, colors thereof, a degree of overlapping between such objects, and the like.

Another objective of the present invention is to provide such a pelvic quotient system which may obtain the vaginal and penile quotients in various images and then assess a degree or extent of matching between such quotients. Accordingly, a related objective of this invention is to provide a system capable of matching such quotients primarily based upon sizes of the images, colors thereof, orientations thereof, meanings thereof, and the like.

Another objective of the present invention is to provide such a pelvic quotient system which may obtain the vaginal and penile quotients and assess an optimum penile quotients for a given vaginal quotient (or vice versa). A related objective of the present invention is to provide a system capable of identifying one or more penile quotients which best match the given vaginal quotient. Another related objective of this invention is to instead provide a system capable of identifying one or more postures for the given vaginal quotient which best match the given penile quotient. Another related objective of this invention is to provide a system capable of identifying multiple postures which best match both of the vaginal and penile quotients and arranging such postures in a sequence easily performed by the user.

Another objective of the present invention is to provide such a pelvic quotient system which may obtain one or more vaginal quotients and multiple penile quotients and match the vaginal quotients with one or more penile quotients (or vice versa). A related objective of this invention is to provide a system capable of generating a list of penile quotients in an ascending or descending order of such a degree of matching therebetween. Another related objective of this invention is to provide a system capable of generating such lists based on a category (e.g., dynamic patterns or configurations of the pelvic or penile structure).

Another objective of the present invention is to provide such a pelvic quotient system which may obtain the vaginal and penile quotients through an internet and enable users to assess matching their quotients with quotients of others. A related objective of this invention is to provide a system capable of generating various lists described in the above paragraph.

Another objective of the present invention is to provide such a pelvic quotient system which may obtain such vaginal and penile quotients and develop an audio program and/or a visual program with which the user may improve her or his quotient. A related objective of this invention is to provide a system capable of obtaining such quotients, assessing the above matching therebetween, and then developing such programs for the purpose of improving the degree of matching therebetween.

Various aspects and/or embodiments of various systems, methods, and/or processes of this invention will now be described, where such aspects and/or embodiments only represent different forms. Such systems, methods, and/or processes of this invention, however, may also be embodied in many other different forms and, therefore, should not be limited to the aspects and/or embodiments which are set forth herein. Rather, various exemplary aspects and/or embodiments described herein are provided so that this disclosure will be thorough and complete, and fully convey the scope of the present invention to one of ordinary skill in the art. It is to be understood that various movements and mechanisms therefor as well as various control algorithms of the prior art devices as described in the above Background of the Invention are to be incorporated herein in their entireties by reference.

In one aspect of the present invention, a system may be provided for assessing at least one quotient representing physiologic states of a pelvic structure including an entry and a wall, where the entry may be arranged to define therethrough an orifice and where the wall may be arranged to have muscles and to form an internal cavity extending inwardly and bound by such muscles. Such a pelvic structure will now be referred to as a “standard pelvic structure” or simply as a “standard structure” throughout this description.

In one exemplary embodiment of this aspect of the invention, a system may include at least one body member and at least one control member. The body member may be arranged to include at least one first unit capable of contacting at least a portion of the pelvic structure when the body member is engaged with the structure. Such a body member is to be referred to as the “body member of the first type” throughout this description. The control member may be arranged to include at least one sensor unit and at least one control unit, where the sensor unit may be arranged to be coupled to the first part and to measure at least one variable representing the state, while the control unit may be arranged to be operatively coupled to the sensor unit. Such a control member is to be referred to as the “control member of the first type” throughout this description. In one example, the control unit may also obtain a value of the variable and assess the quotient directly from the value. In another example, the control unit may obtain a value of the variable, normalize the value by a preset reference, and assess such a quotient from the normalized value. In yet another example, the control unit may obtain a value of such a variable, define a mathematical equation which may be a function of the variable, and assess such a quotient by plugging the value of the variable into the equation.

In another exemplary embodiment of this aspect of the invention, a system may include at least one body member of the first type and at least one control member which may be arranged to include multiple sensor units and at least one control unit. The sensor units may be arranged to be coupled to the first part and to monitor multiple variables each of which may represent different characteristics of the state, whereas the control unit may be arranged to operatively couple with the sensor units and to obtain values of such variables. Such a control member is to be referred to as the “control unit of the second type” throughout this description. In one example, such a control unit may further assess the quotient directly from the values. In another example, the control unit may assess the quotient through mathematically manipulating at least one of the above values. In another example, the control unit may normalize at least one of the values by at least one preset reference, and assess the quotient from such values at least one of which may correspond to the normalized value. In another example, such a control unit may obtain at least one mathematical equation which may be a function of at least one of the variables, and assess the quotient by plugging at least one of the values into the equation.

In another exemplary embodiment of this aspect of the invention, a system may include at least one body member of the first type and at least one control member of the first type whose control unit may also obtain multiple values of the variable by measuring the variable by the sensor unit in each of multiple portions of the structure one at a time. In one example, the control unit may further assess the quotient as a set of at least two of the values of the different portions. In another example, the control unit may normalize at least one of the values by a preset reference and assess the quotient as a set of at least two of the values at least one of which may be the normalized value. In another example, the control unit may obtain at least one mathematical equation which may be a function of at least one of the variables, assess at least one another value by plugging at least one of the values into such an equation, and assess such a quotient as a set of at least two of the values at least one of which may correspond to the assessed value.

In another exemplary embodiment of this aspect of the invention, a system may include at least one body member of the first type and at least one control member of the second type whose control unit may also obtain values of the variables. In one example, the control unit may assess the quotient as a set of at least two of such values of the different portions. In another example, the control unit may be able to mathematically manipulate at least one of the values and assess the quotient as a set of at least two of the values at least one of which is the manipulated value. In another example, the control unit may normalize at least one of the values by at least one preset reference and assess the quotient as a set of at least two of the values at least one of which may correspond to the normalized value. In another example, the control unit may obtain at least one mathematical equation which may be a function of at least one of the variables, assess at least one another value by plugging at least one of such values into the equation, and assess the quotient as a set of at least two of the values at least one of which may correspond to the assessed value.

In another aspect of the present invention, a system may further be provided for assessing at least one quotient which may be a set of multiple values and which may represent physiologic states of the standard pelvic structure.

In one exemplary embodiment of this aspect of the invention, a system may include at least one body member of the first type and at least one control member of the first type. In one example, such a control unit may obtain multiple values of the variable by measuring the variable by the sensor unit in multiple portions of the structure one at a time and assess the quotient as a set of at least two of such values measured in the different portions. In another example, the control unit may also obtain multiple values of the variable by measuring the variable with the sensor unit in multiple postures of the pelvic structure one at a time and assess the quotient as a set of at least two of the values measured in the different postures. In another example, the control unit may obtain multiple values of the variable by measuring the variable with the sensor unit adjusted in multiple baselines one at a time and assess the quotient as a set of at least two of the values which may be measured at the different baselines. In another example, the control unit may obtain multiple values of the variable by measuring the variable while moving at least one of the sensor unit and structure at different speeds one at a time, and then assess the quotient as a set of at least two of the values measured in the different portions.

In another exemplary embodiment of this aspect of the invention, a system may include at least one body member of the first type as well as at least one control member. The control member may be arranged to have multiple sensor units and at least one control unit, where each of the sensor unit may be arranged to be coupled to the first part and to measure multiple variables, while the control unit may be arranged to be operatively coupled to such sensor units, to obtain values of the variables, and to assess such a quotient as a set of at least two of the values. In one example, at least two of such variables for the quotient may be of the same type measured in different portions of the structure. In another example, at least two of the variables for the quotient may be of different types measured in the portion. In another example, at least two of the variables for the quotient may be of different types measured in different portions of the structure. In yet another example, at least one of such variables for the quotient may be arranged to be related to a dynamic pattern of the structure, while at least one another of the variables therefor may be arranged to be related to a configuration of the structure.

In another aspect of the present invention, a system may further be provided for assessing at least one of an audible and visible quotient and representing physiologic states of the standard pelvic structure.

In one exemplary embodiment of this aspect of the invention, a system may include at least one body member of the first type and at least one control member of the first type whose control unit may obtain at least one value of the variable, and to express the quotient as audible signals. Such values may be optionally arranged to be expressed as a tone, a pitch, and/or a melody of the signals. Such values may be optionally arranged to be expressed as a song selected from multiple songs depending upon such a value.

In another exemplary embodiment of this aspect of the invention, a system may include at least one body member of the first type and at least one control member of the first type whose control unit may also obtain multiple values of the variable by measuring the variable by the sensor unit in multiple portions of the pelvic structure one at a time and express such a quotient as a two-dimensional shape and/or a three-dimensional shape. Such values may be optionally arranged to be expressed along at least one axis of the shape and/or to be expressed as a height and/or a thickness of the shape. The values may optionally be arranged to be expressed as protrusions and/or indentations of the shape. In addition, such values may optionally be expressed as a color of at least a section of the shape.

In another exemplary embodiment of this aspect of the invention, a system may include at least one body member of the first type and at least one control member of the second type whose control unit may also obtain values of such variables, and express the quotient as a two-dimensional shape and/or a three-dimensional shape. Such values of one of such variables may be optionally expressed in at least one axis of the shape and/or expressed as two of a height, thickness, length, and/or width of the shape. Such values thereof may optionally expressed as protrusions and/or indentations of the shape. Such values of at least one of the variables may optionally be expressed as a color of at least a section of the shape.

In another aspect of the present invention, another system may be provided to assess multiple quotients each describing physiologic states of the standard pelvic structure.

In one exemplary embodiment of this aspect of the invention, a system may include at least one body member of the first type and at least one control member of the first type whose control unit may also obtain a value of the variable in each of multiple postures of the structure and assess each of the quotients from the value in each of the postures. In one example, the control unit may provide an user with such quotients and postures corresponding thereto. In another example, such a control unit may provide an user with multiple postures in each of which such a quotient may be arranged to exceed a preset threshold.

In another exemplary embodiment of this aspect of the invention, a system may include at least one body member of the first type and at least one control member of the first type whose control unit may also obtain a value of the variable in each of multiple postures of the structure, assess each of the quotients from the value in each of such postures, and obtain at least one male quotient of a male. In one example, the control unit may provide an user with at least one of the quotients and at least one of corresponding postures which may match the male quotient beyond a preset threshold according to a preset standard. In another example, the control unit may provide an user with multiple postures in each of which the quotient may be arranged to match the male quotient beyond a preset threshold according to a preset standard.

In another exemplary embodiment of this aspect of the invention, a system may include at least one body member of the first type and at least one control member of the first type. The control unit may obtain a value of the variable in each of multiple postures of the structure, assess each of such quotients from the value measured in each of the postures, and provide an user with at least one male quotient of male which may match the quotients for the structure beyond a preset threshold according to a preset standard.

In another aspect of the present invention, a system may be provided to measure at least one variable for the standard pelvic structure of a female as well as at least another variable for a penile structure of a male.

In one exemplary embodiment of this aspect of the invention, a system may include at least one control unit which may be arranged to obtain values of such variables for both of such structures, to assess a female quotient based on the value of the pelvic structure, to assess a male quotient based on the value of the penile structure, and to analyze such female and male quotients for assessing a match therebetween.

In another exemplary embodiment of this aspect of the invention, a system may include at least one control unit which may be arranged to obtain values of the variables for both of the structures, to respectively assess a female quotient and a male quotient from the values of the pelvic structure and penile structure, and to assess a degree of matching between the female and male quotients primarily based upon signs of such quotients, magnitudes of the quotients, a similarity between magnitudes of the quotients, a difference between magnitudes of the quotients, at least one of a sum, a product, and a ratio of the quotients, and the like.

In another exemplary embodiment of this aspect of the invention, a system may include at least one control unit which may be arranged to obtain values of the variables for both of the structures, to express each of a female quotient and male quotient as a set of multiple numbers, and to analyze the sets of numbers for assessing a degree of matching between the female and male quotients,

In another exemplary embodiment of this aspect of the invention, a system may include at least one control unit which may be arranged to obtain values of the variables for both of the structures, to express each of a female quotient and a male quotient as a set of multiple numbers, and to assess a degree of matching between the female quotient and male quotient. Such numbers of the female and male quotients may be obtained from the values of the pelvic and penile structures, respectively, and may be arranged in a preset order in order form a profile of magnitudes thereof in such an order. The control unit may then arranged to assess the degree of the matching primarily based upon a variety of criteria. In one example, the criteria may be a sign of one of the numbers of the female quotient and a sign of a corresponding number of the female quotient. In another example, the criteria may instead be a magnitude of one of the numbers of the male quotient and that of a corresponding number of the female quotient. In another example, the criteria may instead be at least one of a sum, a product, and a ratio of one of the numbers of the male quotient and a corresponding number of the male quotient. In another example, the criteria may be a similarity between the profiles of the quotients in the direction or a difference between the profiles of the quotients in the direction. In another example, the criteria may be a complementarity between the profiles of the quotients in the direction.

In another exemplary embodiment of this aspect of the invention, a system may include at least one control unit which may be arranged to obtain values of the variables for both of the structures, to express each of a female quotient and male quotient as audible signals, and to analyze such audible signals for assess a degree of matching between the female and male quotients.

In another exemplary embodiment of this aspect of the invention, a system may include at least one control unit which may be arranged to obtain values of the variables for both of the structures, to express each of a female quotient and male quotient as audible signals, and then to assess a degree of matching between the female and male quotients primarily based upon a variety of criteria such as, e.g., amplitudes of the audible signals, tones thereof, pitches thereof, melodies thereof, content bases thereof, and the like.

In another exemplary embodiment of this aspect of the invention, a system may include at least one control unit which may be arranged to obtain values of the variables for both of the structures, to represent each of a female quotient and a male quotient into a two-dimensional shape and/or a three-dimensional shape, and to analyze the shapes for assessing a degree of matching between the male and female quotients.

In another exemplary embodiment of this aspect of the invention, a system may include at least one control unit which may be arranged to obtain values of the variables for both of the structures, to represent each of a female quotient and a male quotient into a two-dimensional shape and/or a three-dimensional shape, and then to assess a degree of matching between the female and male quotients primarily based upon a variety of criteria such as, e.g., sizes of the shapes., at least one of lengths, heights, widths, and thicknesses of the shapes, a similarity (or congruence) between the shapes, a difference between the shapes, an angle defined between the shapes when overlapped onto each other, and the like.

In another exemplary embodiment of this aspect of the invention, a system may include at least one control unit which may be arranged to obtain values of the variables for both of the structures, to represent each of a female quotient and a male quotient into a two-dimensional shape and/or a three-dimensional shape each forming a preset number of holes based upon the variable, and to assess a degree of matching between such female and male quotients primarily based upon numerous criteria such as, e.g., sizes of the holes defined in the above shapes, numbers of such holes defined therein, locations of the holes in each of the shapes, a degree of overlapping between the holes therein, an angle defined between the shapes when a preset number of the holes may be overlapped onto each other, and the like.

In another exemplary embodiment of this aspect of the invention, a system may include at least one control unit which may be arranged to obtain values of the variables for both of the structures, to represent each of a female quotient and a male quotient as a two-dimensional shape and/or a three-dimensional shape each forming a preset number of protrusions and/or indentations based upon the variable, and to assess a degree of matching between the female and male quotients primarily based upon various criteria. In one example, the criteria may be sizes of the protrusions and/or indentations defined in the shapes. In another example, such criteria may be numbers of such protrusions and/or indentations defined in the shapes. In another example, the criteria may instead be locations of such protrusions and/or indentations defined in the shapes. In another example, the criteria may also be a degree of matching between at least one protrusion of one of the shapes and at least one indentation of the other of the shapes. In yet another example, the criteria may be an angle defined between the shapes when the protrusions and/or indentations of the shapes may be aligned with each other.

In another exemplary embodiment of this aspect of the invention, a system may include at least one control unit which may be arranged to obtain values of the variables for both of the structures, to express each of a female quotient and a male quotient in a monochromic color or a multichromic color, and to analyze both of such colors for assessing a degree of matching between the female and male quotients.

In another exemplary embodiment of this aspect of the invention, a system may include at least one control unit which may be arranged to obtain values of the variables for both of the structures, to represent each of a female quotient and a male quotient in a monochromic color or multichromic color, and then to assess a degree of matching between the female and male quotients primarily based on a numerous criteria such as, e.g., hue of each of the colors of the shapes, brightness thereof, contrast between the colors of the shapes, another color obtained by mixing the colors of the shapes, another color obtained by overlapping the shapes, and the like.

In another exemplary embodiment of this aspect of the invention, a system may include at least one control unit which may be arranged to obtain values of the variables for both of the structures, to represent each of a female quotient and a male quotient as a pattern which includes multiple geometric objects, and to analyze the patterns for assessing a degree of matching between the female and male quotients.

In another exemplary embodiment of this aspect of the invention, a system may include at least one control unit which may be arranged to obtain values of the variables for both of the structures, to represent each of a female quotient and a male quotient as a pattern which includes multiple geometric objects, and to assess a degree of matching between the quotients primarily based upon a variety of criteria such as, e.g., shapes of the objects of the patterns, colors thereof, a degree of overlapping between the objects of the patterns, and the like.

In another exemplary embodiment of this aspect of the invention, a system may include at least one control unit which may be arranged to obtain values of the variables for both of the structures, to represent each of a female quotient and a male quotient as at least one image connoting at least one meaning, and to analyze the meanings for assessing a degree of matching between such female and male quotients.

In another exemplary embodiment of this aspect of the invention, a system may include at least one control unit which may be arranged to obtain values of the variables for both of the structures, to represent each of a female quotient and a male quotient as at least one image connoting at least one meaning, and to assess a degree of matching between the quotients primarily based upon numerous criteria such as, e.g., sizes of the images, colors thereof, orientations thereof, types of the meanings of the images, and the like.

In another aspect of the present invention, another system may be provided so as to measure multiple variables for multiple standard pelvic structures of multiple females and multiple variables for multiple penile structures of multiple males and of matching the females with the males.

In one exemplary embodiment of this aspect of the invention, a system may include at least one control unit which may be arranged to obtain values of the variables for multiple pelvic structures and penile structures, to assess multiple female quotients based on the values from the pelvic structures, to assess multiple male quotients based upon the values of the penile structures, and then to analyze the female quotients and male quotients for assessing at least one match between at least one of the female quotients and male quotients.

In another exemplary embodiment of this aspect of the invention, a system may include at least one control unit which may be arranged to obtain values of the variables for multiple standard pelvic structures and the penile structures through an internet, to assess multiple female quotients based on the values of the pelvic structures, to assess multiple male quotients based on the values of the penile structures, and to analyze the female and male quotients for assessing at least one match between at least one of the female quotients and male quotients.

In another aspect of the present invention, a system may be provided to assess at least one quotient which represents physiologic states of the standard pelvic structure and to provide an user with at least one of an audible program and a visual program for improving such a quotient.

In one exemplary embodiment of this aspect of the invention, a system may include at least one control unit which may be arranged to obtain multiple values of the variable in multiple postures of the pelvic structure, to assess multiple quotients based upon the values, and to provide the user with at least one of the programs which may be arranged to include at least one of the postures in which the quotient assessed therein may exceed a preset threshold.

In another exemplary embodiment of this aspect of the invention, a system may include at least one control unit which may be arranged to obtain multiple values of the variable in multiple postures of the pelvic structure, to assess multiple female quotients based upon the values, to obtain at least one male quotient, and to provide the user with at least one of the programs which may then be arranged to include at least one of the postures in which a degree of matching between such male and female quotients may exceed a preset threshold.

Embodiments of such apparatus aspects of the present invention may include one or more of the following features.

The body member may also define a second unit for providing a grip to the user. Such a part of the first unit may be designed to engage with the clitoris, G spot, and the like.

The variable may be at least one of a normal force exerted by the portion of such a structure, a bending force exerted thereby, an axial force exerted thereby to pull (or push) an object (or the first unit) into and/or out of the internal cavity, torque exerted thereby, contact between the object and the portion of the structure, and the like. The variable may be at least one one of a normal force applied onto at least a part of the first unit, a bending force applied onto the part, an axial force pulling and/or pushing the part (or an object) into and/or out of the internal cavity, a torque applied around (or with respect to) the part, a velocity of the part, an acceleration thereof, a displacement thereof, a contact between such a part and such a portion of the structure, a dimension of the portion, contraction and relaxation of the portion, a duration of at least one of the variables, a frequency of at least one of the variables, and the like. The variable may be at least one of a normal force applied onto the portion of the structure, a bending force applied onto the portion, an axial force resisting movement of an object (or the first unit) into and/or out of the cavity of the structure, a torque applied about (or with respect to) the portion, a velocity of such a portion, an acceleration thereof, a displacement thereof, a contact between the portion and part, contraction and/or relaxation of the portion, a duration of at least one of the variables, a frequency of at least one of the variables, and the like.

The variable may define the dynamic pattern which may include at least one temporal pattern and at least one spatial pattern, and the sensor unit may be arranged to measure (or monitor) at least one of the temporal pattern and spatial pattern. Such a temporal pattern may include at least one of an instantaneous value of the variable, its time-varying value, its time-averaged value, its average which may be weighted by a preset weighting function, its peak value, its time derivative, its integration over time, and the like. The temporal pattern may be at least one of a duration of the variable, its frequency, its temporal sequence, and the like. The spatial pattern may be at least one of a localized value of the variable in a preset region, a distribution of multiple variables over an area, a space-averaged value of the variable, its global or local peak, its derivative along a preset direction, its integration in at least one direction, and the like. The spatial pattern may include at least one of an amplitude of the variable and its direction. The dynamic pattern may be at least one of a frequency of the variable, its temporal rate of change (or its temporal differentiation), its displacement (or its integral over time) caused thereby, and a compound value obtained by at least one of mathematical manipulation of at least one thereof. The configuration may include at least one of a length of the portion, its height, its radius, its diameter, its width, its curvature, and the like. The variable and/or value may be in an analog or digital format.

The control member may obtain one of such variables and assess at least one another of the variables from the one of the variables. The sensor unit may measure the variable along any direction with respect to a longitudinal axis of the body member. The sensor unit may measure at least one of an absolute value of the variable and a relative (or normalized) value of the variable with respect to a preset value thereof. The sensor unit may measure such a variable after adjusting a baseline thereof. The sensor unit may be defined in its head, trunk, and/or base of such a first unit. The control member may include any number of the sensor units which may be disposed in any arrangements and each of which may measure any of the variables. The body member may include multiple sensor units which may be identical, disposed close to each other, disposed apart from each other, and the like.

Such a portion of the pelvic structure may be a clitoris of the entry, a G spot on the wall, other portions of the wall, and the like. The system may play sound and/or display an image of the variable and/or value with internal audio, visual, and/or audiovisual input and/or output units or with external audio, visual or audiovisual input and/or output devices. The sound and/or image may be generated in response to the value and/or variable effected (or initiated) by the user or may be generated to effect the value and/or variable by the user. Such a system may include at least one of an audio output unit capable of playing the value as audible signals and a visual output unit capable of displaying the value as visual signals. The body member and sensor unit may be provided externally such that the system may only include the control unit which may therefore be disposed away from such an external body member and sensor unit but which may be operatively coupled to the sensor unit through wire and/or wirelessly.

In another aspect, a method may be provided for assessing at least one quotient representing a physiologic state of the standard pelvic structure.

In one exemplary embodiment of this aspect of the invention, the method may include the steps of: contacting at least a portion of the pelvic structure; measure at least one variable representing the state; obtaining a value of the variable; and then assessing the quotient directly from the value. Such assessing may instead be replaced by the steps of: normalizing the value by a preset reference; and assessing the quotient from the normalized value. Such assessing may also be replaced by the step of: obtaining a mathematical equation which is a function of the variable; and assessing the quotient by plugging the value of the variable into the equation.

In another exemplary embodiment of this aspect of the invention, the method may also include the steps of: contacting at least a portion of the pelvic structure; measuring multiple variables each of which may represent different characteristics of the state; obtaining values of the variables; and then assessing the quotient directly from the values. The assessing may instead be replaced by the steps of: mathematically manipulating at least one of the values; and assessing the quotient from the above manipulating. The assessing may be replaced by the steps of: normalizing at least one of the values by at least one preset reference; and assessing the quotient from the values at least one of which is the normalized value. The assessing may further be replaced by the steps of: obtaining at least one mathematical equation as a function of at least one of such variables; and assessing the quotient by plugging at least one of the values into the equation.

In another exemplary embodiment of this aspect of the invention, the method may also include the steps of: contacting at least a portion of the pelvic structure; measuring multiple variables each of which may represent different characteristics of the state in multiple portions of the structure one at a time; obtaining multiple values of the variable by such measuring; and assessing the quotient as a set including at least two of the values of the portions. The assessing may be replaced by the steps of: normalizing at least one of the values by a preset reference; and then assessing the quotient as a set including at least two of the values at least one of which is the normalized value. The assessing may be replaced by the steps of: obtaining at least one mathematical equation as a function of at least one of the variables; assessing at least another value by plugging at least one of the values into such an equation; and assessing the quotient as a set which includes at least two of the values at least one of which is the assessed value.

In another exemplary embodiment of this aspect of the invention, the method may also include the steps of: contacting at least a portion of such a pelvic structure; measuring multiple variables each representing different characteristics of the state; obtaining values of such variables; and assessing the quotient as a set of at least two of the values of the different portions. The assessing may also be replaced by the step of: assessing the quotient as a set of at least two of the values of the above different variables. The assessing may be replaced by the steps of: mathematically manipulating at least one of the values; and assessing such a quotient as a set including at least two of the values at least one of which may be the manipulated value. The assessing may also be replaced by the steps of: normalizing at least one of the values by at least one preset reference; and assessing the quotient as a set including at least two of the values at least one of which may be the normalized value. Such assessing may further be replaced by the steps of: obtaining at least one mathematical equation as a function of at least one of the variables; assessing at least another value by plugging at least one of the values into the equation; and assessing the quotient as a set including at least two of the values at least one of which is the assessed value.

In another aspect, a method may be provided for assessing at least one quotient which may be a set of multiple values and which may depict physiologic states of the standard pelvic structure.

In one exemplary embodiment of this aspect of the invention, the method may include the steps of: contacting at least a portion of the pelvic structure; measuring multiple variables each representing different characteristics of the state in multiple portions of the pelvic structure one at a time; obtaining multiple values of the variable by the measuring; and assessing the quotient as a set including at least two of the values measured in the different portions. The assessing may be replaced by the step of: assessing the quotient as a set having at least two of the values measured in the different postures.

In another exemplary embodiment of this aspect of the invention, the method may also include the steps of: contacting at least a portion of the pelvic structure; measuring such a variable in multiple baselines one at a time; obtaining multiple values of the variable by the measuring; and assessing the quotient as a set having at least two of the values measured at the different baselines.

In another exemplary embodiment of this aspect of the invention, the method may also include the steps of: contacting at least a portion of the pelvic structure; measuring the variable while moving at least one of the sensor unit and structure at different speeds one at a time; obtaining multiple values of the variable by the measuring; and then assessing the quotient as a set having at least two of the values measured in the different portions.

In another exemplary embodiment of this aspect of the invention, the method may include the steps of: contacting at least a portion of the pelvic structure; measuring multiple variables; obtaining values of the variables; and assessing the quotient as a set including at least two of the values of the variables of the same type measured in different portions of the structure. Such assessing may also be replaced by the step of: assessing the quotient as a set including at least two of the values of the variables of different types measured in the portion of the structure. The assessing may be replaced by the step of: assessing the quotient as a set including at least two of the values of the variables of different types measured in different portions of the structure. The assessing may be replaced by the step of: assessing the quotient as a set including at least two of the values of such variables at least one of which may be related to a dynamic pattern of the structure and where at least another of the variables therefor may be related to a configuration of the structure.

In another aspect, a method may also be provided for assessing at least one of an audible and visible quotient representing physiologic states of the standard pelvic structure.

In one exemplary embodiment of this aspect of the invention, the method may include the steps of: contacting at least a portion of the pelvic structure; measure at least one variable representing the state; obtaining at least one value of the variable; and expressing the quotient as audible signals. The expressing may be replaced by the step of: expressing the quotient as audible signals while varying their tone based upon the value. The expressing may be replaced by the step of: expressing such a quotient as audible signals while varying their pitch based upon the value. The expressing may also be replaced by the step of: expressing such a quotient as audible signals while varying their melody based upon the value. The expressing may also be replaced by the step of: expressing the quotient as audible signals of a song selected from multiple songs based upon the value.

In another exemplary embodiment of this aspect of the invention, the method may also include the steps of: contacting at least a portion of the pelvic structure; measuring at least one variable that represents the state by moving the sensor unit to multiple portions of the pelvic structure one at a time; obtaining multiple values of the variable by the measuring; and expressing such a quotient as one of a two-dimensional shape and a three-dimensional shape. Such expressing may also include one of the steps of: representing the values along at least one axis of the shape; representing the values as at least one of a height and thickness of the shape; representing the values as one of protrusions and indentations of the shape; and representing the values as a color of at least a section of the shape.

In another exemplary embodiment of this aspect of the invention, the method may also include the steps of: contacting at least a portion of the pelvic structure; measuring multiple variables each of which represents different characteristics of the state; obtaining values of such variables; and then expressing the quotient as at least one of a two-dimensional shape and a three-dimensional shape. The expressing may include one of the steps of: representing the values of one of the variables along at least one axis of the shape; representing the values of two of such variables as two of a height, thickness, length, and width of the shape; representing the values of at least one of the variables as one of a protrusion and an indentation of the shape; and representing the values of at least one of the variables as a color of at least a section of the shape.

In another aspect, a method may be provided for assessing multiple quotients each describing a physiologic state of the standard pelvic structure.

In one exemplary embodiment of this aspect of the invention, the method may include the steps of: contacting at least a portion of the pelvic structure; measuring at least one variable representing the state; obtaining a value of the variable in each of multiple postures of such a structure; assessing each of the quotients from the value in each of the postures; and providing an user with the quotients and the postures corresponding thereto. The providing may be replaced by the step of: providing an user with multiple the postures in each of which the quotient may exceed a preset threshold.

In another exemplary embodiment of this aspect of the invention, the method may also include the steps of: contacting at least a portion of the pelvic structure; measuring at least one variable that may represent the state; obtaining a value of the variable in each of multiple postures of the structure; assessing each of the quotients from the value in each of the postures; obtaining at least one male quotient of a male; and providing to an user at least one of the quotients and at least one of postures corresponding thereto and matching the male quotient beyond a preset threshold based upon a preset standard. The providing may be replaced by the step of: providing an user with multiple the postures in each of which the quotient may be arranged to match the male quotient beyond a preset threshold according to a preset standard.

In another exemplary embodiment of this aspect of the invention, the method may also include the steps of: contacting at least a portion of the pelvic structure; measuring at least one variable that may represent the state; obtaining a value of the variable in each of multiple postures of the structure; assessing each of the quotients from the value measured in each of the postures; and providing to an user at least one male quotient of male matching the quotients for the pelvic structure beyond a preset threshold according to a preset standard.

In another aspect, a method may also be provided for measuring at least one variable for the standard pelvic structure of a female and measuring at least one variable for a penile structure of a male.

In one exemplary embodiment of this aspect of the invention, the method may include the steps of: obtaining values of the variables for both of the structures; assessing a female quotient based on the value of the pelvic structure; assessing a male quotient based on the value of the penile structure; analyzing both of the female and male quotients; and assessing a match between the quotients from the analyzing.

In another exemplary embodiment of this aspect of the invention, the method may also include the steps of: obtaining values of the variables for both of the structures; assessing a female quotient and a male quotient from the values of the structures; and assessing a degree of matching between the female and male quotients primarily based on at least one of signs of the quotients, magnitudes of the quotients, a similarity between magnitudes of the quotients, a difference between magnitudes of the quotients, and at least one of a sum, a product, and a ratio of the quotients.

In another exemplary embodiment of this aspect of the invention, the method may also include the steps of: obtaining values of the variables for both of the structures; expressing each of a female quotient and male quotient as a set of multiple numbers; analyzing the sets of numbers; and assessing a degree of matching between the female and male quotients from the analyzing.

In another exemplary embodiment of this aspect of the invention, the method may also include the steps of: obtaining values of the variables for both of the structures; obtaining multiple numbers from the values of the pelvic and penile structures; arranging the numbers in a preset order to form a profile of magnitudes thereof in the order; expressing each of a female quotient and male quotient as a set of the numbers; and then assessing a degree of matching between the female and male quotients primarily based on one or more of numerous criteria which may include a sign of one of the numbers of the female quotient and that of a corresponding number of the female quotient, a magnitude of one of the numbers of the male quotient and that of a corresponding number of such a female quotient, at least one of a sum, a product, and a ratio of one of the numbers of the male quotient and a number of the female quotient corresponding thereto, a similarity between the profiles of the quotients in such a direction, a difference between the profiles of the quotients along the direction, and a complementarity between the profiles of the quotients in the direction.

In another exemplary embodiment of this aspect of the invention, the method may also include the steps of: obtaining values of the variables for both of the structures; expressing each of a female quotient and a male quotient as audible signals; analyzing the audible signals; and assessing a degree of matching between the female and male quotients from the analyzing.

In another exemplary embodiment of this aspect of the invention, the method may also include the steps of: obtaining values of the variables for both of the structures; expressing each of a female quotient and male quotient as audible signals; and then assessing a degree of matching between the female and male quotients primarily based upon one or more of numerous criteria which may include amplitudes of the audible signals, tones of the audible signals, pitches of the audible signals, melodies of the audible signals, content bases of the audible signals, and the like.

In another exemplary embodiment of this aspect of the invention, the method may also include the steps of: obtaining values of the variables for both of the structures; expressing each of a female quotient and male quotient as at least one of a two-dimensional shape and three-dimensional shape; analyzing the shapes; and assessing a degree of matching between the female and male quotients from the analyzing.

In another exemplary embodiment of this aspect of the invention, the method may include the steps of: obtaining values of the variables for both of the structures; representing each of a female quotient and a male quotient as one of a two-dimensional shape and a three-dimensional shape; and assessing a degree of matching between the female and male quotients primarily based upon one or more of numerous criteria which may include sizes of such shapes, at least one of lengths, heights, widths, and thicknesses of the shapes, a similarity or congruence between the shapes, a difference between the shapes, an angle defined between the shapes when overlapped onto each other, and the like.

In another exemplary embodiment of this aspect of the invention, the method may include the steps of: obtaining values of the variables for both of the structures; representing each of a female quotient and a male quotient as one of a two-dimensional and three-dimensional shape; providing a preset number of holes in the shapes based upon the variable; and assessing a degree of matching between the female and male quotients primarily based upon one or more of numerous criteria which may include sizes of the holes defined in the shapes, the numbers of the holes defined in the shapes, locations of the holes in each of the shapes, a degree of overlapping between the holes in each of the shapes, an angle defined between the shapes when a preset number of the holes are overlapped onto each other, and the like.

In another exemplary embodiment of this aspect of the invention, the method may also include the steps of: obtaining values of the variables for both of the structures; representing each of a male quotient and a female quotient as one of a two-dimensional shape and a three-dimensional shape; forming a preset number of at least one of protrusions and indentations on the shapes based on the variable; and assessing a degree of matching between the female and male quotients primarily based upon one or more of numerous criteria which may include sizes of the at least one of protrusions and indentations defined in such shapes, numbers of at least one of protrusions and indentations defined therein, locations of at least one of protrusions and indentations defined in such shapes, a degree of matching between at least one protrusion of one of such shapes and at least one indentation of the other of such shapes, an angle defined between the shapes when at least one of the protrusions and indentations of the shapes are aligned with each other, and the like.

In another exemplary embodiment of this aspect of the invention, the method may also include the steps of: obtaining values of the variables for both of the structures; expressing each of a female quotient and male quotient in one of a monochromic color and a multichromic color; analyzing both of the colors; and assessing a degree of matching between the female quotient and male quotient from the analyzing.

In another exemplary embodiment of this aspect of the invention, the method may also include the steps of: obtaining values of the variables for both of the structures; representing each of a male quotient and a female quotient in one of a monochromic color and a multichromic color; and assessing a degree of matching between the female quotient and male quotient primarily based on one or more of numerous criteria which may include hue of each of the colors of the shapes, brightness thereof, contrast between the colors of the shapes, another color obtained by mixing the colors of the shapes, another color obtained by overlapping the shapes, and the like.

In another exemplary embodiment of this aspect of the invention, the method may also include the steps of: obtaining values of the variables for both of the structures; representing each of a male quotient and a female quotient as a pattern including multiple geometric objects; analyzing the patterns; and assessing a degree of matching between the female and male quotients from the analyzing.

In another exemplary embodiment of this aspect of the invention, the method may also include the steps of: obtaining values of the variables for both of the structures; representing each of a male quotient and a female quotient as a pattern including multiple geometric objects; and then assessing a degree of matching between the quotients primarily based on one or more of numerous criteria which may include shapes of the objects of the patterns, colors thereof, a degree of overlapping between the objects of the patterns, and the like.

In another exemplary embodiment of this aspect of the invention, the method may also include the steps of: obtaining values of the variables for both of the structures; representing each of a male quotient and a female quotient as at least one image connoting at least one meaning; analyzing such meanings; and assessing a degree of matching between such quotients from the analyzing.

In another exemplary embodiment of this aspect of the invention, the method may also include the steps of: obtaining values of the variables for both of the structures; representing each of a male quotient and a female quotient as at least one image connoting at least one meaning; and assessing a degree of matching between the quotients primarily based on one or more of numerous criteria which may include sizes of the images, colors thereof, orientations thereof, types of the meanings of such images, and the like.

In another aspect, a method may also be provided for measuring multiple variables of multiple standard pelvic structures of multiple females and multiple variables of multiple penile structures of multiple males and of matching the females with the males.

In one exemplary embodiment of this aspect of the invention, the method may include the steps of: obtaining values of the variables for multiple the pelvic structures and multiple the penile structures; assessing multiple female quotients based upon the values of the pelvic structures; assessing multiple male quotients based on the values of the penile structures; analyzing the female quotients and male quotients; and assessing at least one match between at least one of the female quotients and at least one of the male quotients from the analyzing.

In another exemplary embodiment of this aspect of the invention, the method may also include the steps of: obtaining values of the variables for multiple the pelvic structures and multiple the penile structures through an internet; assessing multiple female quotients based on the values of the pelvic structures; assessing multiple male quotients based on the values of the penile structures; analyzing the female and male quotients; and assessing at least one match between at least one of the female quotients and at least one of the male quotients from the analyzing.

In another aspect, a method may be provided for assessing at least one quotient representing physiologic states of the standard pelvic structure including an entry and a wall and providing an user with at least one of an audible program and a visual program for improving the quotient.

In one exemplary embodiment of this aspect of the invention, the method may include the steps of: obtaining multiple values of the variable in multiple postures of such a structure; assessing multiple quotients based upon the values; and providing the user with at least one of the programs which may include at least one of the postures in which the quotient assessed therein exceed a preset threshold.

In another exemplary embodiment of this aspect of the invention, the method may also include the steps of: obtaining multiple values of the variable in multiple postures of the structure; assessing multiple the female quotients based on the values; obtaining at least one male quotient; and providing the user with at least one of the programs which is arranged to include at least one of the postures in which a degree of matching between the male and female quotients exceed a preset threshold.

Embodiments of such method aspects of the present invention may include one or more of the following features.

The method may include the step of: providing a grip to the user. The obtaining may include the step of: engaging a clitoris, a G spot, and other portions of the structure. Such obtaining may include the step of: measuring a normal force exerted by the structure, a bending force exerted thereby, an axial force exerted thereby to pull or push an object (or the first unit) into or out of the internal cavity, torque exerted thereby, contact between the object and the structure, and the like. The obtaining may also include the step of: measuring a velocity of at least a portion of such a structure, an acceleration thereof, a displacement thereof, a contact with the portion of the structure, a dimension of the portion, contraction and relaxation of the portion, a duration of at least one of the variables, a frequency of at least one of the variables, and the like. The obtaining may also include the step of: measuring at least one dynamic pattern of the variable which may include at least one temporal pattern and at least one spatial pattern, where the temporal pattern may include at least one of an instantaneous value of the variable, its time-dependent value, its time-averaged value, its average which may be weighted by a preset weighting function, its peak value, its time derivative, its integration over time, a duration of the variable, its frequency, its temporal sequence, and the like, and where the spatial pattern may include at least one of a localized value of such a variable in a preset portion of the structure, a distribution of multiple variables over an area of the structure, a space-averaged value of the variable, its global or local peak, its derivative along a preset direction, its integration in at least one direction, an amplitude of the variable and its direction, and the like. The obtaining may include the step of: measuring at least one dynamic pattern which may include at least one of a frequency of the variable, its temporal rate of change (or its temporal differentiation), its displacement, its integral over time caused thereby, and a compound value obtained by at least one of mathematical manipulation of at least one thereof. Such obtaining may include the step of: measuring at least one configuration may be at least one of a length of a portion of the structure, its height, its radius, its diameter, its width, its curvature, and the like.

The obtaining may include the step of: measuring the variable and value in an analog or digital format. The obtaining may include the steps of: obtaining one of the variables; and assessing at least one another of the variables from the one of the variables. The obtaining may also include the step of: measuring the variable along any direction. The obtaining may further include at least one of the steps of: measuring an absolute value of the variable; and measuring a relative (or normalized) value of the variable with respect to a preset value thereof. The obtaining may further include at least one of the steps of: maintaining the posture during the obtaining; changing the postures (or generating movement of the pelvic structure) during the obtaining; and contracting and relaxing other muscles of at least one of an abdomen, a back, a waist, a leg, and a thigh of the user. The obtaining may include one of the steps of: contracting and relaxing the muscles of the structure while keeping the posture during such obtaining; varying the posture without relaxing and contracting the muscles during the obtaining; and contracting and relaxing the muscles while changing the posture during the obtaining. The obtaining may include the steps of: adjusting a baseline; and measuring the variable after the adjusting. Such obtaining may include one of the steps of: performing exercise in a pace which may be selected by the user; conforming the exercise to an audible sound; and conforming the exercise to a visual image. The method may also include at least one of the steps of: playing sounds related to the variable; and displaying images related to the variable.

Various product-by-process claims may be constructed by modifying the foregoing preambles or their modifications of the above system and/or method claims and by appending thereto the above bodies or their modifications of the above system and/or method claims. Such process claims may be arranged to include one or more of the aforementioned features of the above system and/or method claims of the present invention.

As used herein, a term “pelvic structure” refers to an anatomic structure of sexual organs of a female. Such a “pelvic structure” typically defines an entry and a wall, where the entry is arranged to define an orifice therethrough, and where the wall is arranged to include various muscles and to also define an internal cavity which is arranged to extend inwardly and to be also bound by the muscles As used herein, both of the terms “sensor unit” and “input unit” refer to those units of various pelvic quotient systems capable of receiving various variables of the pelvic structure (or user inputs) and/or monitoring dynamic pattern thereof. However, the “sensor unit” and the “input unit” are to be differentiated as follows within the scope of this invention. First, such a “sensor unit” is preferably disposed in an insertable part of a body member, while the “input unit” is generally disposed in a part of the body member of the system which is intended to not be inserted into the internal cavity of such a pelvic structure. Therefore, the “sensor unit” generally receives the user inputs (more specifically, various variables related to dynamic patterns and/or configurations of the pelvic structure or simply “pelvic variables”) through various portions of the pelvic structure, whereas the “input unit” primarily receives the user inputs through a hand or a finger of an user.

The terms “proximal” and “distal” will be used in a relative context. Throughout this invention, the term “proximal” is to be used to denote a direction toward a head of a body member of a system, whereas the term “distal” is to be used to represent an opposite direction toward an end of a handle of such a system. Accordingly, a “proximal” end and a “distal” end may be defined with respect to an entire pelvic quotient system or with respect to a specific member or unit thereof.

The terms “input unit” and “sensor unit” generally refer to identical or similar articles capable of monitoring various dynamic patterns of various user inputs applied thereto. Throughout this invention, however, the “input units” represent such articles incorporated into a handle part (or a second unit) of a body member of a pelvic quotient system, while the “sensor units” denote such articles incorporated into an insertable part (or a first unit) of such a body member. Accordingly, any articles which may be used as the “input unit” may also be used as the “sensor unit” unless otherwise specified. In addition, the “input unit” may also be disposed in the insertable part of the body member, while the “sensor unit” may also be disposed in the handle part thereof when desired.

As used herein, a “dynamic pattern” refers to a temporal pattern as well as a spatial pattern of a variable (i.e., a “pelvic variable”), of an user input, and/or of a sensing signal each of which may be generated by a sensor unit and/or an input unit of a control member in response to various variables (or user inputs), while a “dynamic feature” refers to a temporal feature as well as a spatial feature of movement of a single part or multiple parts of a body member.

Unless otherwise defined in the following specification, all technical and scientific terms used herein have the same meaning as commonly understood by one of ordinary skill in the art to which the present invention belongs. Although the methods or materials equivalent or similar to those described herein can be used in the practice or in the testing of the present invention, the suitable methods and materials are described below. All publications, patent applications, patents, and/or other references mentioned herein (particularly those enumerated in the above Background section) are incorporated by reference in their entirety. In case of any conflict, the present specification, including definitions, will control. In addition, the materials, methods, and examples are illustrative only and not intended to be limiting.

Other features and advantages of the present invention will be apparent from the following detailed description, and from the claims.

### BRIEF DESCRIPTION OF THE DRAWING

FIGS. 1A to 1D are partially cutaway schematic views of exemplary pelvic quotient systems including various sensor units according to the present invention;

FIGS. 2A to 2D are schematic views of exemplary pelvic quotient systems with various body members according to the present invention;

FIGS. 3A to 3L are schematic views of exemplary emblems representing various quotients for a pelvic structure according to the present invention; and

FIG. 4 is a schematic diagram of various functional members and units of an exemplary pelvic quotient system according to the present invention.

### DETAILED DESCRIPTION OF THE PREFERRED EMBODIMENTS

The present invention generally relates to systems for assessing quantitative quotients related to various static and dynamic characteristics of pelvic (i.e., vaginal) structures of females and penile structures of males associated with sexual activities. More particularly, the present invention relates to various systems capable of measuring one or multiple variables of such structures each of which represents a physiologic state of such structures, obtaining values of such variables, and assessing various sexual quotients such as vaginal quotients for females and penile quotients for males. Such a system may include at least one sensor unit for measuring such variables and at least one control unit for processing sensing signals generated by the sensor unit and assessing such quotients according to a preset format. Such a system may have a single sensor unit but dispose such an unit in multiple portions of the pelvic structure for measuring multiple values of a single variable. Such a system may instead include multiple sensor units and measure different variables in a single or multiple portions of the pelvic structure. The control unit may assess the quotients in various formats such as, e.g., pure numbers, numbers and symbols (including alphabets), a set of numbers and/or symbols, and the like. The system may also assess such quotients in sounds, images, and tangible articles such as various emblems. The present invention also relates to another system capable of measuring corresponding variables of the penile structure and assessing corresponding quotients. The present invention also relates to various systems capable of matching such quotients of females with those of males based upon various formats.

The present invention relates to various methods of assessing such quotients from the values of various variables and matching a given vaginal quotient with one of multiple penile quotients (or vice versa). The present invention also relates to various methods of measuring the pelvic variables using a single or multiple sensor units, measuring such variables in a single or multiple portions of the pelvic structure, and assessing the quotients from the values of such variables. The present invention also relates to further methods of generating such quotients by a single number and/or symbol, by a set of multiple numbers and/or symbols, providing such quotients in sounds and/or images, embodying such quotients in the tangible articles, and the like. The present invention further relates to various methods of matching such quotients of the pelvic structures of females with those of the penile structures of males (or vice versa). The present invention also relates to various processes for producing various members, units, and/or parts of the above system and providing various formats of such quotients.

Various aspects and/or embodiments of various systems, methods, and/or processes of this invention will now be described more particularly with reference to the accompanying drawings and text, where such aspects and/or embodiments thereof only represent different forms. Such systems, methods, and/or processes of this invention, however, may also be embodied in many other different forms and, accordingly, should not be limited to such aspects and/or embodiments which are set forth herein. Rather, various exemplary aspects and/or embodiments described herein are provided so that this disclosure will be thorough and complete, and fully convey the scope of the present invention to one of ordinary skill in the relevant art.

Unless otherwise specified, it is to be understood that various members, units, elements, and parts of various systems of the present invention are not typically drawn to scales and/or proportions for ease of illustration. It is also to be understood that such members, units, elements, and/or parts of various systems of this invention designated by the same numerals may typically represent the same, similar, and/or functionally equivalent members, units, elements, and/or parts thereof, respectively.

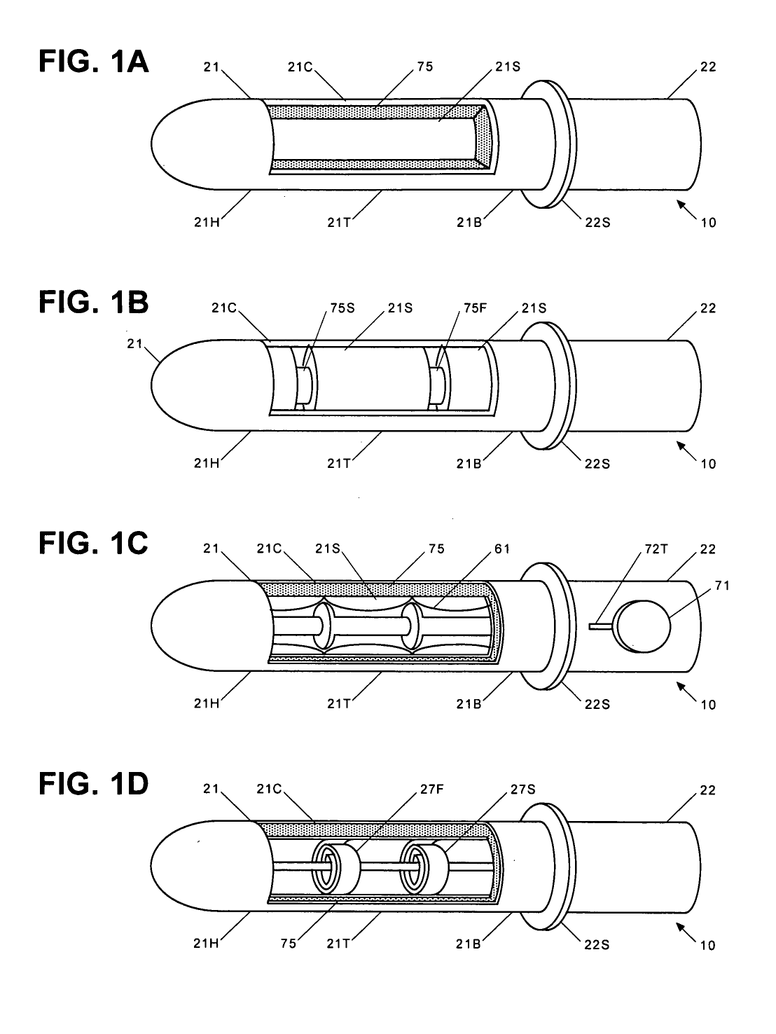

In one aspect of the present invention, pelvic quotient systems may be provided with various sensing mechanisms for measuring and monitoring various variables representing physiologic states of a pelvic structure of an user and assessing vaginal quotients therefrom. FIGS. 1A through 1D are partially cutaway schematic views of exemplary pelvic quotient systems including a single or multiple sensor units capable of measuring the same or different variables of the pelvic structure according to the present invention.

In one exemplary embodiment of such an aspect of the invention and as described in FIG. 1A, an exemplary pelvic quotient system 10 includes a body member 20 with a first unit 21 and a second unit 22 as well as a control member with a sensor unit 75, where such a system 10 may also include other members and/or units as disclosed in the co-pending Applications. The first unit 21 is generally elongated and arranged to be inserted into the pelvic cavity during exercise, while the second unit 22 is distally coupled to the first unit 21 and arranged to form a grip for an user during exercise. The first unit 21 is divided into a head 21H, a trunk 21T, and a base 21B. The head 21H is formed in a proximal end of the body member 20 and rounded at its tip to facilitate insertion of the body member 20 into the pelvic cavity, while the trunk 21T is connected distally to the head 21H and terminates in the base 21B which may couple with the second unit 22. The head 21H, trunk 21T, and base 21B may generally be fixedly or movably coupled to one another and form an unitary body member 20. In the alternative, the head 21H, trunk 21T, and base 21B may instead be arranged to form an unitary first unit 21. As far as the user may monitor a physiologic state of her pelvic structure during exercise, detailed construction of the body member 20 and coupling modes of their parts 21H, 21T, 21B may not be generally material to the scope of the present invention. The second unit 22 is also elongated and has a shape and size to form the grip for the user, whether the user grabs the second unit 22 with his or her thumb pointing proximally, distally, and/or laterally. The second unit 22 may movably or fixedly couple with each other and form an unitary body member 20. In the alternative, the first and second units 21, 22 may instead define an unitary body member 20. A stop 22S is also defined between the first and second units 21, 22 and extends beyond exteriors of such units 21, 22. The stop 22S is generally shaped and sized to prevent the first unit 21 from being inserted into the internal cavity of such a pelvic structure beyond a desirable distance and/or depth. It is appreciated, however, that the stop 22S may be incorporated in any location along the first and/or second units 21, 22 depending on configurations of those units 21, 22 and a maximum desired penetration depth of the first unit 21 into the internal cavity of the structure. As far as the user may monitor such physiologic states of the pelvic structure, detailed construction of and/or coupling modes between such first and second units 21, 22 are not material to the scope of the present invention. As will be described below, the pelvic quotient system 10 also includes other members and units for normal operation. When the first and/or second units 21, 22 may form cavities therein, such members and/or units may then be incorporated into one or both of such units 21, 22. In the alternative, at least one of such members and/or units may be exposed through surfaces of such units 21, 22.

The first unit 21 also includes at least one cover 21C and at least one support 21S, where the cover 21C encloses the rest of the first unit 21 therein, and where the support 21S forms a backbone of the first unit 21. The cover 21C may be made of and/or include at least one inert material such that insertion of the first unit 21 into the pelvic structure may not cause any undesirable reactions by such pelvic walls of the structure. The cover 21C may also be made to be flexible or rigid, depending upon whether various physiological variables may have be transmitted therethrough toward the sensor unit 75 when such a unit 75 may be disposed thereunder. The cover 21C may be shaped and/or sized to enclose an entire part of the first unit 21 or, in the alternative, to enclose only a selected part thereof. In addition, the cover 21C may be arranged to enclose different parts of the first unit 21 by an uniform thickness or varying thicknesses. Depending upon configuration of the first unit 21, such a cover 21C may be arranged to be symmetric with respect to a point and/or line of the first unit 21 or, alternatively, to be asymmetric. As long as the cover 21C may physically isolate an interior of the first unit 21 from the pelvic structure when engaged therewith, detailed shapes and/or sizes of the cover 21C may not be material to the scope of the present invention.

The support 21S is typically disposed inside the first unit 21 in order to provide the first unit 21 with a desired configuration. Depending on design considerations, such a support 21H may be made of and/or include at least one rigid or flexible material. An exact dimension of the support 21 typically depends upon many factors such as, e.g., a dimension of the first unit 21, desired flexibility or rigidity of the first unit 21, and the like. Such a support 21S may also assist the sensor unit 75 to monitor the pelvic variables within a preset accuracy. To this end, the support 21S may be arranged to define a preset rigidity as will be described in greater detail below.

At least one sensor unit 75 may then be incorporated between the cover 21C and support 21H so that various physiological variables of the pelvic structure may be transmitted to the sensor unit 75 through the rigid cover 21C. In the embodiment shown in FIG. 1A, the cover 21C may enclose at least a substantial part of the first unit 21 and the support 21H may extend across at least a substantial part of the first unit 21 while defining an annular gap therebetween. The sensor unit 75 may be arranged to fill the gap and to receive the physiological variables of the pelvic structure through the cover 21C disposed thereover and therearound. The sensor unit 75 is also arranged to change its configuration or to deform at least a part thereof while varying its electrical resistance in response to force applied thereto, where examples of such sensor units 75 may include, but not be limited to, conductive foams with a preset porosity, conductive elastic polymers with a preset porosity or those without including any pores, and so on. It is appreciated that the system 10 of this embodiment has a single sensor unit 75 which is disposed along the substantial part of the first unit 21.

Although not shown in the figure, the system 10 may include other members and/or unit which may output to the user values of various physiologic variables of the pelvic structure monitored by the sensor unit 75. For example, the system 10 may include at least one audio and/or visual output unit so that the values of the monitored pelvic variables may be provided to the user audibly as sounds and/or visually as images. More details of such output units will be described in greater detail below. Other configurational details of such a system 10 of FIG. 1A may be similar or identical to various systems of the co-pending Applications.

In operation, the first and second units 21, 22 are fixedly or movably coupled to each other to form the body member 20. The stop 22S may be disposed between the first and second units 21, 22. The system 10 is then connected to an electric power outlet with a power supply cable (not included in the figure) or provided with a battery disposed inside the first or second unit 21, 22. The user may insert the first unit 21 of the body member 20 into a desirable depth into the internal cavity of the pelvic structure and start exercise such as, e.g., contracting and relaxing the pelvic muscles while keeping a preset posture of the pelvic structure, changing the posture of the pelvic structure by moving her legs or thighs or bending her back, and so on. When the first unit 21 is inserted into the internal cavity, the conductive foam of the sensor unit 75 begins to deform in response to a normal force applied thereto by the pelvic muscles and/or wall. Such a change in configuration of the sensor unit 75 changes the electrical resistance thereof. The control member may measure the change in the electrical resistance and assess the physiologic states of the pelvic structure from various dynamic patterns of the force applied to the sensor unit 75 through the pelvic structure. Depending upon a system configuration, the control member may output various audible and/or visual signals such that the user may monitor such physiological states of her pelvic structure. It is appreciated that such a system 10 of this embodiment includes only a single sensor unit 75 and, therefore, that the change in the electrical resistance of the sensor unit 75 may be regarded as a physiologic value representing the force which is averaged over an area in which the sensor unit 75 is disposed, across which the force is applied, and so on. Other operational details of such a system 10 of FIG. 1A may be similar or identical to various systems of the co-pending Applications.

In another exemplary embodiment of such an aspect of the invention and as shown in FIG. 1B, an exemplary pelvic quotient system 10 includes a body member 20 with a first unit 21 and a second unit 22 as well as a control member all of which are similar to those of FIG. 1A. Accordingly, the first unit 21 forms the head 21H, trunk 21T, and base 21B, and also couples with the second unit 22. The first unit 21 also forms the cover 21C, while the stop 22S is disposed along the first or second unit 21, 22 to prevent the first unit 21 from being inserted into the internal cavity of the pelvic structure beyond a desirable distance and/or depth.

Inside the first unit 21 are incorporated multiple supports 21S which may similar or identical to those of FIG. 1A and may similarly be rigid or flexible. Each adjacent pair of the supports 21S may be coupled to each other by at least one sensor unit 75 of the control member, where such a sensor unit may be arranged to measure displacement or deformation of one of such supports 21S with respect to the other, to measure a force causing rotation or pivoting of one of such supports 21S with respect to the other, and the like. Because such sensor units 75 are arranged to measure the forces exerted onto different parts of the first unit 21, such sensor units 75 may then assess a bending force which is a result of multiple misaligned forces or which is a result of the force applied onto one end of such a first unit 21 which is fixedly supported by another end thereof. In the embodiment shown in FIG. 1B, each of three supports 21S is respectively disposed in the head 21H, trunk 21T, and base 21B of the first unit 21, and each of two sensor units 75 is disposed between the head 21H and trunk 21T of the first unit 21 and between the trunk 21T and base 21B thereof. It is appreciated that such sensor units 75 may measure such displacement or forces without resulting any deformation thereof or may do so while deforming at least a part thereof. Because such sensor units 75 may only have to measure the displacement or deformation of one support 21S with respect to the other or to monitor forces applied onto different parts of the first unit 21, such sensor units 75 may not have to be in direct contact with the pelvic structure, cover 21C of the first unit 21, and the like, as long as various pelvic variables may be transmitted to such supports 21S. In the alternative, the sensor units 75 may instead be disposed on the cover 21C of the first unit 21, inside the cover 21C or immediately below the cover 21C. When desirable, the first unit 21 may include more (or less) supports 21S and more (or less) sensor units 75 may be incorporated to measure the bending force. It is appreciated that multiple sensor units 75 may allow the user to monitor a spatial distribution of such bending forces across various parts of the first unit 21. Such sensor units 75 may be disposed side by side along the longitudinal axis of the first unit 21 or along the circumference thereof, thereby providing different spatial distribution of such bending forces measured along different directions. To the contrary, the control member may include a single sensor unit which may measure an overall or lumped bending force which is deemed to be averaged over a preset area or part of the first unit 21.

Although not shown in the figure, the system 10 may include other members and/or unit which may output to the user values of various physiologic variables of the pelvic structure monitored by the sensor unit 75. For example, the system 10 may include at least one audio and/or visual output unit so that the values of the monitored pelvic variables may be provided to the user audibly as sounds and/or visually as images. More details of such output units will be described in greater detail below. Other configurational details of such a system 10 of FIG. 1B may be similar or identical to various systems of the co-pending Applications.

In operation, the first and second units 21, 22 are fixedly or movably coupled to each other to form the body member 20. The stop 22S may be disposed between the first and second units 21, 22. The system 10 is then connected to an electric power outlet with a power supply cable (not included in the figure) or provided with a battery disposed inside the first or second unit 21, 22. The user may insert the first unit 21 of the body member 20 into a desirable depth into the internal cavity of the pelvic structure and start exercise such as, e.g., contracting and relaxing the pelvic muscles while keeping a preset posture of the pelvic structure, changing the posture of the pelvic structure by moving her legs or thighs or bending her back, and the like. When the first unit 21 is inserted into the internal cavity of the pelvic structure, different parts of the first unit 21 receive the misaligned forces with different (or similar) amplitudes from the structure and deviate from their normal arrangement. Such displacement, deformation or movement may move or deform such supports 21S in different extents, and the sensor units 75 generate sensing signals. The control member receives and analyzes such signals and then assess various dynamic patterns of such displacement, deformation or force resulting in the bending of the first unit 21 and/or sensor units 75. Depending on a system configuration, the control member may output various audible and/or visual signals so that the user may monitor the physiological states of her pelvic structure. Other operational details of the system 10 shown in FIG. 1B may be similar or identical to those of FIG. 1A and/or those of various systems of the co-pending Applications.

In another exemplary embodiment of this aspect of the invention and as described in FIG. 1C, an exemplary pelvic quotient system 10 includes a body member 20 with a first unit 21 and a second unit 22, all of which are typically similar to that of FIG. 1A. Therefore, the first unit 21 forms the cover 21C, head 21H, trunk 21T, and base 21B, the second unit 22 forms the grip for the user, and the stop 22S is disposed along the first or second unit 21, 22. The system 10 also includes a control member with at least one sensor unit 75 which is disposed between the cover 21C and support 21S such that various physiological variables of the pelvic structure may be transmitted to the sensor unit 75 through the rigid cover 21C. In the embodiment of FIG. 1C, the sensor unit 75 may be arranged to maintain its configuration. Thus, the sensor unit 75 does not change its shape and/or size during measurement of various pelvic variables. The sensor unit 75 of this embodiment may employ any of the above sensing mechanisms.

The support 21S may form protrusions and indentations and at least one driver unit 61 may be disposed inside and along the first unit 21. More particularly, the driver unit 61 may define protrusions and indentations matching those of the support 21S and move or translate along a longitudinal axis of the first unit 21. Thus, the protrusions of the driver unit 61 may be disposed between the indentations of the support 21S when the driver unit 61 is disposed distally, while the protrusions of the driver unit 61 may abut such indentations of the support 21S when the driver unit 61 may be pushed proximally. The second unit 22 includes at least one input unit 71 which may be arranged to move along a track 72T which is also defined along the second unit 22. Such an input unit 71 may be operatively coupled to the driver unit 61 so that movement of the input unit 71 may move and/or translate the driver unit 61 proximally or distally. It is appreciated that the sensor unit 75 of such an embodiment may maintain its configuration during the movement of the driver unit 61. Other configurational details of the system 10 of FIG. 1C may also be similar or identical to those of FIGS. 1A and 1B and/or those of other systems of the co-pending Applications.

In operation, the first and second units 21, 22 are fixedly or movably coupled to each other to form the body member 20, and the stop 22S may be disposed between the first and second units 21, 22. In this rest position, the protrusions of the driver unit 61 may be trapped in or near the indentations of the support 21S, thereby manipulating the first unit 21 to maintain a low profile. After the system 10 is connected to power, the user may insert the first unit 21 into a desirable depth into the cavity of the pelvic structure. The user may manipulate the input unit 71 along the track 72T and translate the driver unit 61 with respect to the support 21S so that the protrusions of the driver unit 61 may gradually abut the protrusions of the support 21S, thereby gradually increasing the profile of the first unit 21. As the sensor unit 75 may snugly fit onto the pelvic wall and touch the pelvic muscles, the baseline may then be adjusted. Depending upon operating mechanisms thereof, the sensor unit may have to be adjusted so that the sensor unit with a new configuration may issue new baseline sensing signals, particularly when such a sensor unit 75 may preferably measure the pelvic variables in absolute values. Once the sensor unit 75 may be fitted onto the pelvic structure and/or contact the pelvic structure, the user may start the pelvic exercise such as, e.g., contracting and relaxing the pelvic muscles while keeping a preset posture of the pelvic structure, varying the posture of the pelvic structure by moving her legs or thighs or bending her back, and so on. The sensor unit 75 may begin to issue the sensing signals, and the control member may measure desirable pelvic variables representing the physiologic states of the structure from various dynamic patterns thereof. Depending upon a system configuration, such a control member may output various audible and/or visual signals so that the user may monitor various physiological states of her structure. Further operational details of the system 10 of FIG. 1C may also be similar or identical to those of FIGS. 1A and 1B and/or those of various systems of the co-pending Applications.

In another exemplary embodiment of this aspect of the invention and as described in FIG. 1D, an exemplary pelvic quotient system 10 includes a body member 20 with a first unit 21 and a second unit 22 and a control member with a sensor unit 75, where the system 10 is typically similar to that of FIG. 1A. Accordingly, the first unit 21 defines the cover 21C, head 21H, trunk 21T, and base 21B, the second unit 22 forms the grip for the user, and the stop 22S is disposed along the first or second unit 21, 22. At least one sensor unit 75 may also be disposed between the cover 21C and support 21H so that various physiological variables of the pelvic structure may be transmitted toward the sensor unit 75 through the rigid cover 21C.

Such a system 10 may also include multiple load units 27F, 27S which are generally disposed inside the first unit 21. In the embodiment of FIG. 1D, the load units 27F, 27S may include substantially elastic (or resistant) elements such as, e.g., coils or springs, which are disposed side by side along a longitudinal axis of the first unit 21. In addition, such elastic (or resistant) load units 27F, 27S may also operatively couple to the cover 21C and sensor unit 75 so that such units 27F, 27S may pose a preset elastic (or resistant) load to the pelvic structure during pelvic exercise. Because such load units 27F, 27S are disposed parallel to each other, the pelvic structure may be posed by an elastic (or resistant) load which may be a sum of elastic (or resistant) loads posed by each of such units 27F, 27S. When desirable, the system 10 may include a viscous load unit as descried in the co-pending Applications. Other configurational and/or operational details of the system 10 of FIG. 1D may be similar or identical to those of FIGS. 1A to 1C and/or those of various systems of such co-pending Applications.

Configurational and/or operational variations and/or modifications of the above embodiments of the exemplary systems and various members thereof described in FIGS. 1A through 1D also fall within the scope of this invention.

Such sensor units may be arranged to measure various pelvic variables. For example, such a sensor unit may measure changes in lengths of the first and/or sensor units caused by displacement or deformation of such units, changes in surface areas or cross-sectional areas of the units caused thereby, changes in volumes of such units caused thereby, changes in an angle defined between at least two preset parts of such units, changes in curvature along at least a part of such units, and the like. It is appreciated that any pelvic variables defined in any directions may be measured as long as the sensor units may be properly aligned to measure such variables related to bending of such a part of the first or sensor unit. Alternatively, the control member may be arranged to assess such pelvic variables from different variables measured by such sensor units. Further details of this embodiment will be provided below.

In addition to various sensor units disclosed in FIGS. 1A to 1D, other conventional sensors may also be incorporated into the sensor units and measure other pelvic variables, where examples of the conventional sensors may include, but not be limited to, force sensors (or force transducers), velocity sensors (or velocimeters), acceleration sensors (or accelerometers), displacement sensors capable of measuring the above variables associated with displacement or deformation of at least a part of the first and/or sensor units, contact sensors employing mechanical, electrical, magnetic, and/or chemical mechanisms, duration or timing sensors such as clocks or timers, electric current or voltage sensors (or meters) for measuring physiologic current or voltage associated with contraction and relaxation of the pelvic muscles, and the like. Any of these sensor units may then be disposed in preset locations of the first unit and in preset arrangements in order to measure various pelvic variables defined along a preset direction.

The sensor units may measure the pelvic variables in absolute values, and the control member may provide such values to the user for monitoring purposes. In the alternative, the sensor units may measure such variables in absolute values, and the control member may then convert the values into relative values by normalizing the absolute values with respect to various preset reference values of the same or different variable, where examples of such preset reference values may include, but not be limited to, a preset value of the same (or different) variable determined by a manufacturer or user, a preset value of the same (or different) variable averaged over a preset time interval and/or a preset area, a peak or maximum (or minimum) value of the same (or different) variable, a value of the same (or different) variable obtained in a proceeding measurement, and so on. Such values may further be obtained in an analog format or a digital format. In another alternative, the sensor units may measure the variables in one or more of the above relative values, where the control member may provide such relative values to the user. In addition and as described hereinabove, the sensor units and/or control member may measure and provide such absolute and/or relative values of the pelvic variables to the user without performing the baseline adjustment or after adjusting such a baseline.

The control member may include any number of such sensor units which may be disposed in almost any arrangements and which may measure the same or different variables which are defined in the same or different portions of the pelvic structure. In one example, the control member may have at least two sensor units which may monitor the same variable and may be disposed in different parts of the first unit. In another example, the control member may include at least two sensor units which may be disposed in a preset part of the first unit and measure different variables. In another example, the control member may include at least two sensor units which may be disposed in different parts of the first unit and measure different variables. When desirable, the sensor units may be disposed one over the other or side by side. In addition, at least one of such sensor units may also be disposed in the second unit and/or stop defined along the first or second unit.

As described above, such sensor units may be defined in the head, trunk, and/or base of the first unit. When the system includes multiple sensor units, at least two of such units may be identical, similar or different, at least two of such units may be disposed close to each other, away from each other, or one above the other.

The system may play sound and/or display an image of the variable and/or value thereof with an internal audio output unit, an internal visual output unit, and/or an internal audiovisual output unit. In case of storing the signals, the system may also include an internal audio input unit, an internal visual input unit, and/or an internal audiovisual input unit. Such sound and/or image may then be generated in response to the measured or assessed value of the variable which may be effected or initiated by the user or may be generated to effect such a variable with such a value by the user.

Such sensor units may be disposed in preset strategic locations along the first or second unit in order to measure the variables defined in, on or around a clitoris of the entry of the pelvic structure, a G spot on the pelvic wall, other portions of the wall, and the like.

Each of such pelvic variables may also define dynamic patterns which may be one or both of temporal patterns and spatial patterns, where examples of the temporal pattern may include, but not be limited to, an instantaneous value of the variable, a time-varying (or time-dependent) value thereof, a time-averaged value thereof, an average thereof weighted by a preset weighting functions, a peak value thereof, a time derivative thereof in the first, second or higher order, an integration thereof over time, and the like, while examples of the spatial pattern may include, but not be limited to, a localized value of the variable, a spatial distribution thereof, an area-averaged value thereof, its global or local peak in a preset domain (e.g., a preset area or volume), a spatial derivative thereof in the first, second or higher order, a spatial derivative thereof along one or more directions, an integration over a preset length, area or volume, and the like.

The temporal patterns of such pelvic variables may also include a duration of such a variable, its frequency, its temporal sequence, and the like, and the spatial patterns may include an amplitude of the variable, its direction, and the like. The dynamic pattern may further include a frequency of such a variable, its temporal rate of change (or temporal differentiation), its displacement (or its integral over time) caused thereby, and a compound value obtained by at least one of mathematical manipulation of at least one thereof. In addition, the dynamic pattern may include a duty cycle of any of the variables such as, e.g., periods in which such variables may change their amplitudes and/or direction, areas in which such variables may be defined, and the like.

In addition, such variables may include normal force applied onto at least a part of the first unit, bending force applied to such a part, axial force pulling or pushing the part into (or out on the internal cavity, torque applied around the part, velocity of the part, acceleration of such a part, displacement of the part, contact between the part and a corresponding portion of the pelvic structure, a dimension of the portion, contraction and relaxation of the portion, a duration of at least one of such variables, a frequency of at least one of such variables, and so on. In the alternative, such variables may include normal force applied onto at least a portion of the pelvic structure, bending force applied to the portion, axial force resisting movement of the first unit into (or out of) the cavity of the pelvic structure, torque applied around such a portion, velocity of the portion, acceleration of the portion, displacement of the portion, contact between such a portion and such a part, contraction and relaxation of such a portion, a duration of at least one of such variables, a frequency of at least one of the variables, and the like. Such force may also be a torque effected about an axis of rotation or pivoting of at least a part of the sensor unit and/or at least a portion of the structure. It is appreciated that such sensor units including the force sensors may be made of and/or include elastic and/or deformable materials so as to deform in response to such force. In the alternative, such sensor units may maintain its configuration during measuring the pelvic variables by employing rigid sensors such as, e.g., piezoelectric sensors.

Each of such temporal and/or spatial patterns (i.e., the dynamic pattern) of the variable may be directly measured by the sensor unit or, alternatively, may instead be assessed from other measured patterns, where such assessment may then be performed directly by the sensor unit or by the control member. For example, the sensor unit may be the force transducer (or pressure sensor) capable of measuring various forces or pressures applied thereto or applied onto at least a portion of the pelvic structure, while the control member may assess therefrom acceleration of the sensor unit and/or the portion of the pelvic structure, velocity of the part of the sensor unit and/or of the portion of the pelvic structure, displacement of the part and/or portion, mass of the part and/or portion, momentum of such a part and/or portion, mechanical energy associated with the part and/or portion, duration of the force (or pressure), frequency of the force (or pressure), and the like. In another example, the sensor unit may be the displacement sensor capable of measuring extents of the deformation or displacement of at least a part of the sensor unit and/or at least a portion of the structure, and the control member may assess therefrom acceleration of such a part and/or portion, velocity of the part and/or portion, mass of the part and/or portion, force (or pressure) applied to the part and/or portion, momentum associated with such a part and/or portion, mechanical energy associated with the a part and/or portion, duration of the movement of such a part and/or portion effecting such displacement or deformation, frequency of such movement, and the like.

Still referring to the same variation or modification, the sensor unit may be the velocity sensor capable of measuring the velocity of at least a part of the sensor unit and/or at least a portion of the pelvic structure, while the control member may assess therefrom acceleration of such a part and/or portion, displacement of the part and/or portion, mass of the part and/or portion, force (or pressure) applied onto of the part and/or portion, momentum associated with the part and/or portion, mechanical energy associated with the part and/or portion, duration of movement of the part and/or portion which may cause such a velocity, frequency of such movement of the part and/or portion, and the like. It is appreciated that the sensor unit may measure or assess the distance to the pelvic structure, velocity of the part and/or portion, and the like, by measuring the distance to the structure. In another example, the sensor unit may also be the acceleration sensor capable of measuring the acceleration of at least a part of the sensor unit and/or at least a portion of the pelvic structure, and the control member may assess therefrom velocity of such a part and/or portion, displacement of the part and/or portion, mass of the part and/or portion, force (or pressure) applied to the part and/or portion, momentum related to the part and/or portion, mechanical energy related to the part and/or portion, duration of movement of such a part and/or portion which effects such acceleration, frequency of the movement, and the like. In another example, the sensor unit may be any conventional sensor capable of monitoring electrical, mechanical, magnetic, and/or chemical contact between at least a part of the first unit and at least a portion of the structure, while the control member may assess therefrom a duration of the contact, a frequency of such contact, and so on. When desirable, the sensor unit may include any conventional optical sensors to detect such contact. It is to be understood that such sensor units for detecting the contact may be arranged to operate based upon a preset threshold. Accordingly, the sensor unit for detecting mechanical contact may be arranged to detect such contact only when the sensor unit may be disposed within a preset distance from the pelvic structure, only when the structure exerts force defining an amplitude exceeding the threshold, and the like. Similar provisions may be applied to other sensor units for electrically, magnetically, optically, and/or chemically detecting such contact.

In addition to the above pelvic variables, the sensor units may be arranged to measure a shape and/or size of at least a portion of the pelvic structure. For example, the sensor unit may be arranged to measure the diameter or radius of a preset portion of the structure, the length or depth into a preset portion thereof, and the like. It is preferred, however, that such measurements be performed after the baseline adjustment of the sensor and/or first units so as to measure more accurate dimension of the portion of the structure.

The sensor units may also measure electric voltages and/or currents representing contraction and/or relaxation capabilities of the pelvic muscles. Any conventional voltage and/or current meters may be employed for such purposes. Alternatively, such sensor units may also measure the voltages and/or currents generated by the system and delivered to the pelvic muscles. In this embodiment, the sensor units may also measure other variables of the pelvic structure which are evoked in response to such voltages and/or currents.

Various clocks and/or timers may also be employed as the sensor units and measure various timings and/or durations associated with any of the above pelvic variables. Accordingly, such sensor units for measuring the timings, durations, and/or frequencies may be used in conjunction with other sensor units in order to determine such temporal patterns of such variables.

It is to be understood that such sensor units may measure some of the pelvic variables such as, e.g., displacement or deformation of at least a part of the sensor unit and/or at least a portion of the pelvic structure, velocity of such a part and/or portion, and acceleration of the part and/or portion, in various arrangements. In one example, the sensor units may deform or move while measuring such variables. In another example, the sensor units may instead maintain their shapes and/or sizes while measuring such variables but such sensor units may be incorporated into a preset part of the first unit which may deform or move during such measurements. In another example, both of the sensor and first units may maintain their shapes and/or sizes during such measurements.

As manifest throughout this disclosure, various sensor units of the pelvic quotient systems of this invention aim to measure various variables representing physiologic states of the pelvic structure, where such states may be elicited by, e.g., capabilities of contracting and relaxing the pelvic muscles, configurations of the pelvic structure, changes in such configurations of such a structure in different postures of the structure, changes in the configurations associated with movement of other portions of a body of the user, and the like. Accordingly, such sensor units may further be tailored in order to measure other variables representing any of the above states of the pelvic structure.

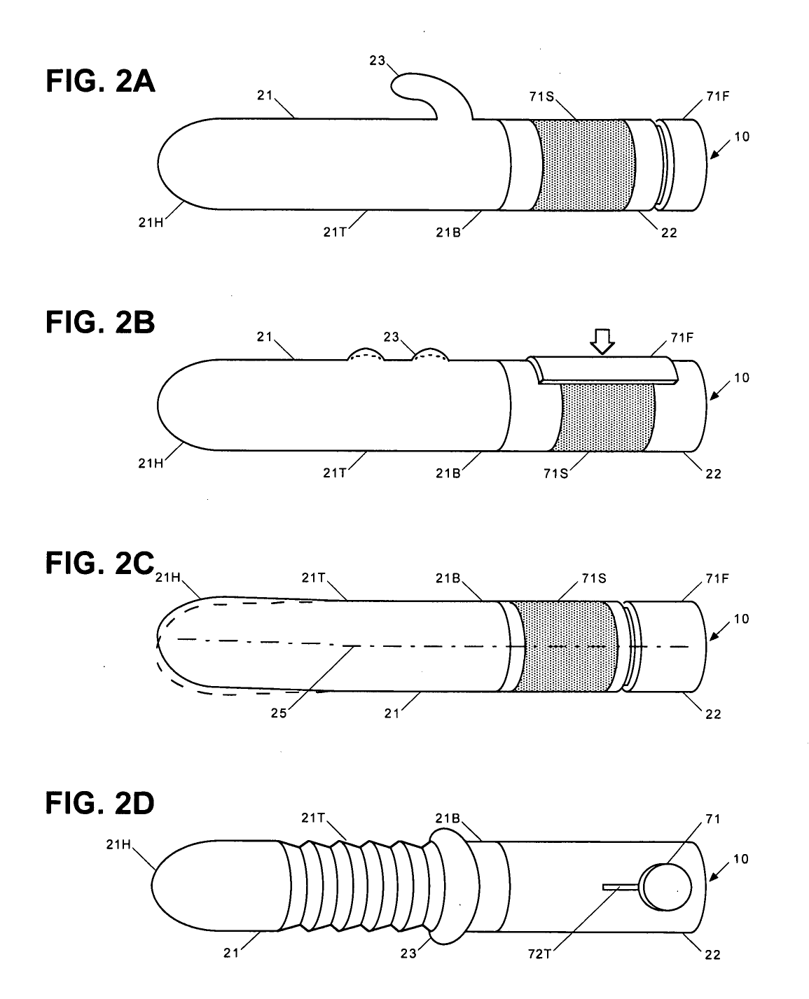

In another aspect of the present invention, the sensor units described above as well as others disclosed in the co-pending Application entitled “Pelvic Exercise Systems” may also be incorporated to other pelvic relaxing systems such as, e.g., “Dynamic Control Pelvic Relaxing Systems,” “Audio Pelvic Relaxing Systems,” and “Synchronized Relaxing Systems,” all of which have already been disclosed in other co-pending Applications. FIGS. 2A to 2D show schematic views of exemplary pelvic quotient systems with various body members according to the present invention.

In one exemplary embodiment of this aspect of the invention and as described in FIG. 2A, an exemplary pelvic quotient system 10 includes a body member 20 with a first unit 21 and a second unit 22, an actuator member incorporated inside the first and second units 21, 22 of the body member 20, and a control member including a first input unit 71F and a second input unit 71B. Although not shown in the figure, the system 10 may be coupled to at least one external audiovisual (to be abbreviated as “A/V” hereinafter) input and/or output units 91, 93 as described in the co-pending Applications. Such a first unit 21 includes a bulge 23 which is formed in the base 21B and which is strategically shaped and/or sized to contact a clitoris of the pelvic structure. Accordingly, an entire part or at least a part of the bulge 23 is arranged to effect vibration and/or other movements to deliver stimuli to the clitoris. When desirable, the bulge 23 may be arranged to translate, rotate, pivot, swivel, vibrate or otherwise move between multiple states while changing its disposition, orientation, and the like, for providing the stimuli to different portions of the structure, providing different stimuli to the same or different portions of the structure, and the like. The bulge 23 may be arranged to deform when abutted by the structure, when manipulated by the control member, and the like.

The first input unit 71F is movably incorporated onto a distal end of the second unit 22 and also oriented so that the user may apply the user input thereto through at least an area of the first input unit 71F vertically, horizontally, at an angle or angularly. The first input unit 71F moves or operates similar to conventional joysticks, and the second input unit 71S is of a variable-resistance type and exposed through the second unit 22. In addition, the control member is arranged to control disposition and/or orientation of the bulge 23 with respect to the rest of the first unit 21 so that manipulating such a first input unit 71F in one direction manipulates the bulge 23 to move or deform along the same or opposite direction. The control member may also adjust a height of the bulge 23 when the first input unit 71F is pivoted about the longitudinal axis of the body member 20. The first input unit 71F may manipulate the bulge 23 in various embodiments as well. The sensor units may be incorporated into various parts of the body member 20. For example, such sensor units may be exposed through the exterior of the first unit 21 or disposed thereunder as long as the variables may be transmitted through the exterior. Such sensor units may be disposed on top of, around, adjacent to, and/or inside such a bulge 23 in order to measure various pelvic variables associated with the clitoris and/or opening of the pelvic structure. Other configurational and/or operational characteristics of the system 10 shown in FIG. 2A have been disposed in the co-pending Applications.

In another exemplary embodiment of this aspect of the invention and as depicted in FIG. 2B, an exemplary pelvic quotient system 10 includes a body member 20 with a first unit 21 and a second unit 22, an actuator member incorporated inside the first and second units 21, 22 of the body member 20, and a control member including a first input unit 71F and a second input unit 71B, each of which may be similar or identical to a corresponding member or unit of FIG. 2A. The system 10 may couple with at least one external A/V input and/or output units 91, 93 as described in the co-pending Applications. In contrary to that of FIG. 2A, however, the first unit 21 includes multiple bulges 23 which are included in the base 21B and strategically shaped and/or sized to contact a G-spot of the pelvic structure. The bulges 23 may be arranged to effect vibration, translation, rotation, and/or other movements in order to deliver stimuli to the G-spot. When desirable, the bulges 23 may also be arranged to translate, rotate, pivot, swivel, vibrate, and/or otherwise move between multiple states while changing their disposition, orientation, arrangement, and the like. Accordingly, such bulges 23 may provide the stimuli to different portions of the structure, provide different stimuli to the same or different portions thereof, and so on. The bulges 23 may also be arranged to deform when abutted by the structure, when manipulated by the control member, and the like.

The first input unit 71F is incorporated onto the second unit 22 and oriented such that the user may apply the user input through at least an area of the first input unit 71F vertically, horizontally, at an angle or angularly. The first input unit 71F is preferably arranged to sense a location thereof to which the user applies the user input, e.g., similar to conventional touch pads or touch screens, and then to generate sensing signals which carry information regarding spatial features of such user inputs. The second input unit 71S is exposed through the second unit 22 and operates generally similar to that of FIG. 2A. The control member is arranged to manipulate disposition and/or orientation of the bulges 23 with respect to the rest of the first unit 21. Accordingly, applying the user input onto the first input unit 71F in one direction manipulates the bulges 23 to move or deform in the same (or opposite) direction. Such a control member is arranged to adjust disposition of the bulges 23 as the user input is applied to different areas of the first input unit 71F. Such a first input unit 71F may be arranged to manipulate the bulges 23 in various embodiment as well. The sensor units may be incorporated into various parts of the body member 20. For example, the sensor units may be exposed through the exterior of the first unit 21 or disposed thereunder as long as the variables may be transmitted through the exterior. Such sensor units may also be disposed on top of, around, adjacent to, and/or between such bulges 23 so as to measure various pelvic variables associated with the G-spot and/or other portions of the pelvic structure. Other configurational and/or operational characteristics of the system 10 shown in FIG. 2B have been disposed in the co-pending Applications.

In another exemplary embodiment of this aspect of the invention and as depicted in FIG. 2C, an exemplary pelvic quotient system 10 includes a body member 20 with a first unit 21 and a second unit 22, an actuator member incorporated inside the first and second units 21, 22 of the body member 20, and a control member including a first input unit 71F and a second input unit 71B, each of which may be similar or identical to a corresponding member or unit of FIG. 2A. The system 10 may couple with at least one external A/V input and/or output units 91, 93 as described in the co-pending Applications. In contrary to those of FIGS. 2A and 2B, the first unit 21 may be arranged to change its curvature with respect to a longitudinal axis 25 of the body member 20 such that various parts of the first unit 21 may contact different portions of the pelvic structure and/or may contact such portions in different angles and/or with different magnitudes of force.

The first and second input units 71F, 71S are both incorporated into the second unit 22, similar to those of FIG. 2A. However, the first input unit 71F may be arranged to manipulate such a curvature defined between proximal and distal parts of the longitudinal axis 25 so that moving the first input unit 71F along one direction increases the curvature, whereas moving such a first input unit 71F along an opposite direction smoothens the curvature. Accordingly, pivoting or swiveling the first input unit 71F upwardly may cause the curved proximal part of the first unit 21 to bend upwardly and rotate along another circular path which is also normal or perpendicular to the curved longitudinal axis 25 (or vice versa). The second input unit 71S is exposed through the second unit 22 and operates similar to that of FIG. 2A. Such a control member may also be arranged to adjust disposition of a point of bending of the first unit 21 as the user input is applied to different areas of the first input unit 71F. The first input unit 71F may also be arranged to manipulate the curvature in various embodiment as well. The sensor units may be incorporated into various parts of the body member 20. For example, such units may be exposed through the exterior of the first unit 21 or disposed thereunder as long as the variables may be transmitted through the exterior. Such sensor units may be disposed on top of, around, adjacent to, and/or between such bulges 23 so as to measure various pelvic variables associated with the G-spot, clitoris, and/or other portions of the pelvic structure. Further configurational and/or operational characteristics of the system 10 of FIG. 2C have been disposed in the co-pending Applications.

In another exemplary embodiment of this aspect of the invention and as depicted in FIG. 2D, an exemplary pelvic quotient system 10 includes a body member 20 with a first unit 21 and a second unit 22, an actuator member incorporated inside the first and second units 21, 22 of the body member 20, and a control member including a first input unit 71F and a second input unit 71B. The system 10 may couple with at least one external A/V input and/or output units 91, 93 as described in the co-pending Applications. Contrary to those of FIGS. 2A to 2C, the first unit 21 has an annular bulge 23 between its trunk 21T and base 21B which protrudes outwardly to abut the inner wall of the internal cavity of the structure when inserted thereinto. In addition, a middle part of the first unit 21 is also arranged to translate along the longitudinal axis of the body member 20 with respect to the rest of the first unit 21, while maintaining the lengths of the heat 21H and base 21B thereof. Accordingly, the first unit 21 may change its length as its trunk 21T translates between its off-state (i.e., when the first unit 21 defines a minimum length) and its on-state (i.e., as the first unit 21 translates and/or deforms into a maximum length). Accordingly, the system 10 may vary the length of the inserted part of the first unit 21 during its use while retaining the inserted and translating part of the first unit 21 inside the internal cavity by abutting the internal walls of the pelvic structure with the above bulge 23 of the first unit 21. Although not shown in FIG. 1C, the actuator member is preferably arranged to generate translating movements of the first unit 21 by conventional various mechanisms of converting rotational movement of its driver unit into the translating movements.

The second unit 22 defines therealong at least one track 72T which extends linearly along the longitudinal axis of the body member 20. The input unit 71 is movably disposed inside the track 72T in order to move along the track 72T while being guided thereby. The input unit 71 also defines a circular receiving area and receives through such an area the user input which moves the input unit 72T along the track 72T. Such an input unit 71 is also arranged to manipulate a displacement or a length of travel of the trunk 21T of the first unit 21 between its off-and on-state, e.g., the length of travel is kept to the minimum when the input unit 71 is positioned in a distal end of the track 72T and to the maximum when the input unit 71 is moved to a proximal end of the track 72T. The input unit 71 may define at least one more state between the proximal and distal ends of the track 72T so that the length of travel may take a value between the maximum and minimum. The sensor units may be incorporated into various parts of the body member 20. For example, such units may be exposed through the exterior of the first unit 21 or disposed thereunder as far as the variables may be transmitted therethrough. The sensor units may be disposed on top of, around, adjacent to, and/or between the middle part so as to measure the pelvic variables associated with the G-spot, clitoris, other portions of the structure, and so on. Other configurational and/or operational characteristics of the system 10 of FIG. 2D have been disposed in the co-pending Applications.

In another aspect of the present invention, pelvic quotient systems may be arranged to assess sexual quotients such as vaginal and penile quotients from various sources and/or based on various formats. In general, such quotients may be assessed in any of the following sources and expressed in any of the following formats. In order to perform comparison of multiple quotients as well as to find a degree of matching therebetween, however, it is preferred that the quotients be assessed from an identical source (or similar sources) and expressed in an identical format (or similar formats).

In one exemplary embodiment of this aspect of the invention, the control unit may obtain values of various variables of the pelvic and penile structures and assess such vaginal and penile quotients as a single or multiple numbers and/or a single or multiple symbols which include alphabets of various languages. In one example, the quotient may be expressed as a whole number defining an arbitrary number of digits, a natural number with a proper sign, a real number with or without at least one digit after a decimal point, and the like. Such a quotient may generally be expressed as the number itself. In the alternative, each digit of the number may represent the value of one of multiple variables. When desirable, a special symbol may also be utilized to represent multiple values of multiple variables. For example, a fraction form or a form including a square root sign may be used to represent the values of different variables in a numerator and a denominator or inside and outside such a square root sign. In another example, the quotient may be expressed by a single or multiple symbols, where such symbols may be assembled to form a word with a meaning. When desirable, the quotient may be expressed by multiple groups of symbols which may be separated by other symbols such as a hyphen, a dash, and the like. In another example, the quotient may be expressed by a mixture of at least one number and at least one symbol, where the quotient may be formed according to various formats described in this paragraph.

Based upon these quotients, the control unit may also assess the degree of matching between the vaginal and penile quotients according to numerous criteria. In one example, the matching may be assessed based on the signs of the quotients, magnitudes thereof accounting for the signs (or not), a similarity between such numbers, a difference therebetween, a sum of such numbers, their product, their ratio, and the like. Accordingly, such quotients may be deemed to match each other better when their values are closer to each other, farther apart from each other, when their sum or their product is larger or less, and the like. In the alternative, the quotients may be deemed to match each other better when the quotients may be complementary, i.e., the quotients are added, multiplied or divided closer to a preset number. When the quotient includes the symbol, the matching may further be assessed by an order of such symbols, upper- or lower-case letters, and the like.

In another exemplary embodiment of this aspect of the invention, such a control unit may obtain the values of various variables of the pelvic and penile structures and assess each of the vaginal and penile quotients as a set of numbers or symbols. In one example, such a set may include two or more numbers or symbols, where an order of such numbers or symbols may express a certain variable of the structure. In another example, such a set may include multiple numbers or symbols arranged in a geometrical pattern, where such an arrangement may also connote a certain variable of the structure. The numbers or symbols of each set may then be formed according to the same or similar formats as described in the above two paragraphs.

Based upon these quotients, the control unit may also assess the degree of matching between the vaginal and penile quotients according to numerous criteria. In one example, the matching may be assessed based on the signs of the quotients, magnitudes thereof accounting for the signs (or not), a similarity between such numbers, a difference therebetween, a sum of such numbers, their product, their ratio, and the like. In addition, a profile of such numbers assigned to the set (e.g., an increase or decrease along a preset order) may also be used as an additional criteria in assessing the quotients. When the quotient includes the symbol, the matching may be assessed by an order of such symbols, upper- or lower-case letters, and the like. It is appreciated, however, that the order or arrangements of the numbers or symbols may be given a special consideration so that the first number of the vaginal quotient may preferably be compared with the corresponding first number of the penile quotient, and the like. Therefore, such quotients may be deemed to match each other better when their values with the same position in such sets are closer to each other, farther apart from each other, when their sum or their product is larger or less, and the like. In the alternative, the quotients may be deemed to match each other better when the quotients may be complementary, i.e., the quotients are added, multiplied or divided closer to a preset number. In addition, such quotients may be deemed to match each other better when such numbers of one set are more congruent or similar to those numbers of another set, when such numbers of one set are complementary or opposite to those numbers of another set, and the like. When the quotient includes the symbol, such matching may also be assessed by an order of such symbols, upper- or lower-case letters, and the like.

In another exemplary embodiment of this aspect of the invention, such a control unit may obtain the values of various variables of the pelvic and penile structures and assess each of the vaginal and penile quotients in audible signals. In one example, each quotient may be expressed as a single note sustained over a preset or variable period or as a series of notes such as, e.g., a musical phrase. In another example, each quotient may be defined as a song with or without a lyric. In another example, each quotient may be expressed as audible signals without any specific tone.

Based upon these quotients, the control unit may also assess the degree of matching between the vaginal and penile quotients according to numerous criteria. In one example, the matching may be assessed based on amplitudes or volumes of the quotients, tones thereof, pitches thereof, a similarity between such notes, a difference therebetween, melodies of such quotients, content bases thereof, and the like. Accordingly, such quotients may be deemed to match each other better when their tones or pitches are closer to each other or farther apart from each other, when their melodies are similar to or different from each other, when their content bases may connote similar or identical meanings, and the like.

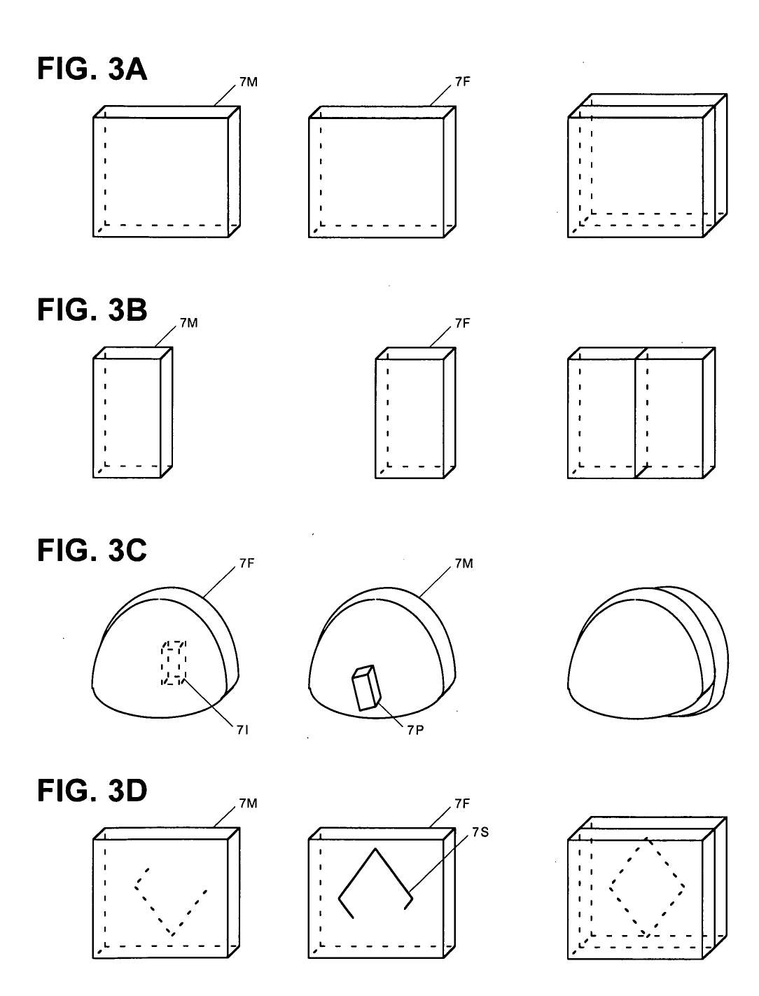

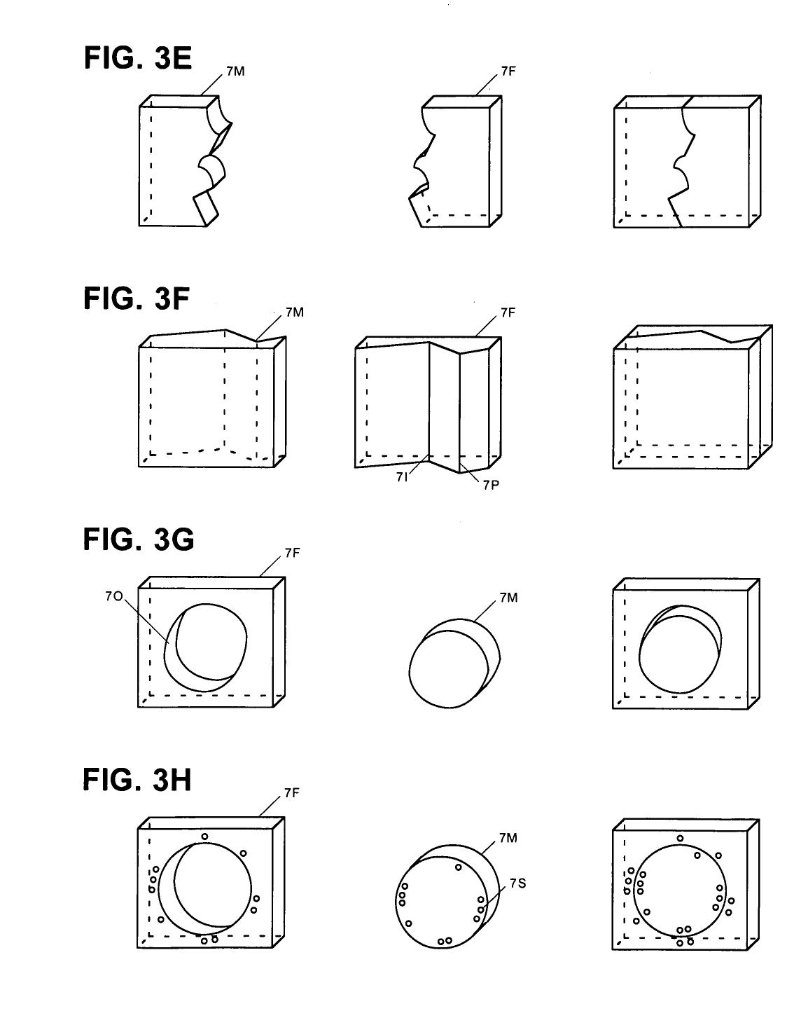

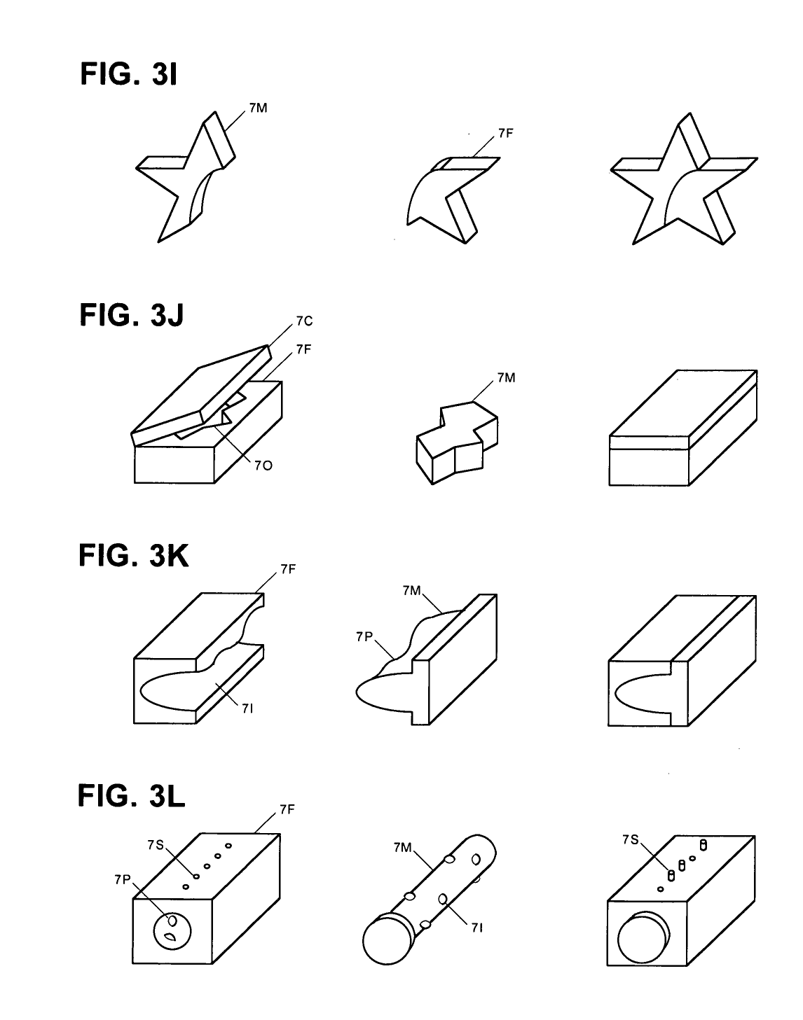

In another exemplary embodiment of this aspect of the invention, such a control unit may obtain the values of various variables of the pelvic and penile structures and assess each of the vaginal and penile quotients in monochromic or multichromic signals, i.e., in gray-scales or colors. In one example, each quotient may be expressed in a single gray-scale or by a single color. In another example, each quotient may be represented by multiple color elements each of which may in turn be represented in a single gray-scale or by a single color. Such color elements may be arranged side by side in a single direction (e.g., similar to a rainbow) or, alternatively, may be arranged in a two- or three-dimensional pattern (e.g., similar to a row or an array of tiles). Such color elements may also be arranged along an emblem which will be described in greater detail in conjunction with FIGS. 3A to 3L.

Based upon these quotients, the control unit may also assess the degree of matching between the vaginal and penile quotients according to numerous criteria. In one example, the matching may be assessed based upon hues of the quotients, brightness thereof, a contrast between such quotients, another gray-scale or color obtained by mixing such quotients, another gray-scale or color obtained by overlapping such quotients, and the like. Therefore, such quotients may be deemed to match each other better when the hues or brightness of such quotients are closer to each other or farther apart from each other, when such quotients are mixed or overlapped closer to a preset gray-scale or color such as black or white (i.e., transparent), and the like.

In another exemplary embodiment of this aspect of the invention, such a control unit may obtain the values of various variables of the pelvic and penile structures and assess each of the vaginal and penile quotients as a pattern including one or more geometric objects. In one example, each quotient may be assessed as a two-dimensional pattern consisting of multiple rows and/or columns of objects such as, e.g., triangles, rectangles, squares, circles or ovals, where each object may have the same shapes, may be assigned with monochromic or multichromic colors, and the like. In another example, such quotients may be assessed as two-dimensional patterns including multiple curvilinear geometric objects which may have identical or different shapes and/or sizes, and may be assigned with colors.

Based upon these quotients, the control unit may also assess the degree of matching between the vaginal and penile quotients according to numerous criteria. In one example, the matching may be assessed based on shapes and/or sizes of the patterns, monochromic or multichromic colors thereof, a similarity in arrangements of such objects in the patterns, a difference in such arrangements, and so on. In another example, the matching may also be assessed by a degree of overlapping between the objects when the patterns are overlapped onto each other, by a degree of continuity between such objects when the patterns are disposed side by side, and the like. Therefore, such quotients may be deemed to match each other better when the shapes and/or sizes of the patterns are closer to each other or deviate more from each other, when various objects are arranged in similar arrangements in such patterns, when such objects are congruent each other as the patterns are overlaid over each other, when such objects arranged along opposing edges of different patterns combine with each other to form a preset image when disposed side by side, and the like.

In another exemplary embodiment of this aspect of the invention, such a control unit may obtain the values of various variables of the pelvic and penile structures and assess each of the vaginal and penile quotients as visible images. In one example, each quotient may be assessed by a shape and/or size of the image, a shape and/or size of a background surrounding the image, ratios of the shape or size of the image to that of the background, a monochromic or multichromic color of the image and/or background, a contrast in such colors of the image and background, and the like. In another example, each quotient may be assessed in terms of orientation of the image with respect to a preset landmark, where such a landmark may correspond to a preset part of the background, a preset part of another article carrying such an image, and the like. In another example, each quotient may be assessed by a meaning connoted by such an image. Therefore, such quotients may be deemed to match each other better when the images of such quotients may have similar shapes, sizes, and/or colors, when such images may be formed in similar orientations, when such images connote similar or identical meanings. Alternatively, such quotients may be deemed to match each other when their images couple with each other to form another image, when the images form another image when overlapped over each other, when such images are complementary to each other, when the meanings carried by such images are similar to or complementary with each other, and the like.

Still referring to the same aspect of the present invention, such pelvic quotient systems may be arranged to assess sexual quotients such as vaginal and penile quotients based on various sources. In one example and when such quotients are expressed in numbers, the values of various variables may be obtained in absolute numbers based on the MKS (meter-kilogram-second) or FPS (foot-pound-second) unit. Alternatively, such values may be obtained as relative values which may not define any dimension, i.e., as dimensionless numbers which may be obtained by calculating a ratio of the variable to a preset reference value of such a variable. Regardless of their units, such variables may further be obtained in various bases, e.g., before or after performing such baseline adjustment as outlined in the co-pending Applications, when the pelvic structure is kept in a preset posture or such a structure changes its posture, when the body member of the system is disposed in a stationary manner around or in a preset portion of the pelvic structure or such a member moves inside the internal cavity of the structure, and the like. In another example for such quotients expressed in numbers, the values of the variables may be substituted into at least one mathematical formula which is represented as a function of at least one of such variables. In this example, such an equation may be developed based upon an anatomy of the pelvic or penile structure or may be obtained empirically based upon statistical results. Such an equation may be a polynomial or any other mathematically solvable formula. When desirable, the mathematical equation may be replaced by a table with multiple entries, where a value of a preset variable may be obtained by an interpolation between two adjacent entries. In another example, such values may be obtained for a single variable which is measured in different states (e.g., static state or dynamic state), in different portions of such structures, in different postures, in different baselines of the sensor unit, and the like. In another example, such values may be obtained for multiple variables belonging to different categories, where each category may include such variables defining the same unit, such variables measured by the sensor units disposed in the same part thereof and/or along the same axial or radial part thereof, such variables measured in a preset posture of the structure, and so on. It is appreciated that those variables of the last two examples may be suitable to form the quotient expressed as a set of multiple numbers as described above. When desirable, various values of such variables may also be replaced by the symbols or may be used with the numbers. In addition, various values of the variables may also be obtained from multiple sources and/or based on multiple formats.

Regardless of its format, it is preferred that each quotient denote certain characteristics of the pelvic and/or penile structures in a quantitative manner. In general, such a quotient may represent at least one of configurational characteristics of such structures, dynamic characteristics thereof, and their sensitivities.

First, such a quotient may be arranged to represent the configurational characteristics of such structures. For example, the quotient may be arranged to represent a dimension of a specific portion of the structure in a preset posture, a change in the dimension in different postures, a change in such a dimension along a direction into (or out on the internal cavity of the pelvic structure or a shaft of the penile structure, and so on. In another example, the quotient may be arranged to represent an overall or averaged dimension of a larger portion or an entire portion of the structure in a posture, a change in the averaged dimension in different postures, and so on. In another example, such a quotient may be arranged to represent a curvature around a specific portion of such a structure in a preset posture, a change in the curvature in different postures, a change in the curvature along a direction into (or out on the internal cavity of the pelvic structure or the shaft of the penile structure, and so on. In another example, the quotient may be arranged to denote an overall or averaged curvature of a larger portion or entire portion of the structure in a preset posture, a change in the averaged curvature in different postures, and the like. In another example, the quotient may further be arranged to represent a spatial distribution of the above dimension and/or curvature along a preset direction such as, e.g., an axial or longitudinal direction defined along the internal cavity of the pelvic structure and/or shaft of the penile structure, a circumferential direction which may be formed perpendicular to and radially with respect to the axial direction, an angular direction about the longitudinal direction, and the like. The quotient may consist of a single number or symbol or, in the alternative, of multiple numbers or symbols which may constitute the set as described above, where the numbers or symbols may represent the above dimensions or curvatures measured in different locations along such a direction. In the latter format, the overall dimension or curvature may become manifest from the profile of such numbers or symbols along the direction. In another example, the quotient may further be arranged to represent a temporal distribution of the above dimension and/or curvature over a preset event such as, e.g., during pelvic exercise, during sexual intercourse, during orgasm, and the like. The quotient may consist of a single number or symbol or, in the alternative, of multiple numbers or symbols which may constitute the set as described above, where the numbers or symbols may represent such dimensions or curvatures measured in preset intervals during such an event. In the latter format, the dimension or curvature of the structure during the event may become manifest from the profile of such numbers or symbols as well. It is appreciated that the overall or averaged dimension or curvature may mean the dimension or curvature averaged over a period of time (i.e., temporally averaged) or over a preset area of such a structure (i.e., spatially averaged). In addition to such dimension and curvature, other configurational characteristics of the structures which have been described in the co-pending Applications may also be expressed by the vaginal and penile quotients when desirable.

Secondly, such a quotient may be arranged to represent the dynamic characteristics of such structures. For example, the quotient may be arranged to denote dynamic patterns of various pelvic or penile variables associated with a specific portion of the structure in a preset posture, a change in such dynamic patterns in different postures, a change in such dynamic patterns in a direction into (or out of the internal cavity of the pelvic structure and/or the shaft of the penile structure, and the like, where details of such dynamic patterns of various pelvic variables have been enumerated above and disclosed in the co-pending Applications, while details of the dynamic patterns of the penile variables are similar or identical to those of the pelvic variables. Accordingly, an exemplary pelvic quotient may relate to an amplitude of the normal (or contracting) force exerted by a preset portion of the structure in a preset posture, a change in the amplitude in different postures of the pelvic structure, and the like. In another example, the quotient may be arranged to represent overall or averaged dynamic patterns of various pelvic or penile variables associated with a specific portion of the structure disposed in a preset posture, a change in such dynamic patterns in different postures, a change in such dynamic patterns in a direction into (or out on the internal cavity of the pelvic structure and/or the shaft of the penile structure, and the like, where such an overall or averaged dynamic pattern may represent the pattern averaged temporally and/spatially. Accordingly, another exemplary pelvic quotient may relate to an averaged amplitude of the normal (or contracting) force exerted by a larger or entire portion of the structure in a preset posture or exerted by at least a portion of the structure over a preset period of time, a change in such an averaged amplitude in different postures of the pelvic structure, and the like. In another example, the quotient may further be arranged to represent a spatial distribution of the above dynamic patterns in a preset direction such as, e.g., the axial or longitudinal direction defined as above, the circumferential direction which may be formed perpendicular to and radially with respect to the axial direction, the angular direction about the longitudinal direction, and the like. The quotient may consist of a single number or symbol or, in the alternative, of multiple numbers or symbols which may constitute the set as described above, where the numbers or symbols may represent such dynamic patterns measured in different locations along the direction. In the latter format, the overall dynamic pattern may become manifest from the profile of such numbers or symbols along such a direction or order. In another example, the quotient may further be arranged to represent a temporal distribution of such a dynamic pattern over a preset event such as, e.g., during pelvic exercise, sexual intercourse, orgasm, and the like. The quotient may consist of a single number or symbol or, in the alternative, of multiple numbers or symbols which may constitute the set as described above, where the numbers or symbols may represent such dynamic patterns measured in preset intervals during such an event. In the latter format, the dynamic patterns of the structure during the event may become manifest from the profile of such numbers or symbols as well. In addition to the above, other dynamic patterns of such structures which have been described in the co-pending Applications may also be expressed by the vaginal and penile quotients, where the dynamic patterns of the penile variables may further include a hardness of the structure during erection, a period of such an erection, a speed of movement of such a structure during sexual intercourse, and the like.

Thirdly, such a quotient may also be arranged to represent the sensitivities of such structures. In one example, such a quotient may be arranged to represent disposition of sensitive portions of the structures such as, e.g., the clitoris and G-spot of the pelvic structure, various nerve clusters of the penile structure, and the like. In general, disposition of such portions may preferably be defined with respect to a preset landmark of such a structure. In another example, such a quotient may represent exposure of such sensitive positions or ease of accessing such portions. As to the clitoris, presence or absence of protruding tissues which tend to cover the clitoris may be included in the quotient, and its shape and/or size may also be accounted for thereby. As to the G-spot, tissues surrounding such a spot may be a critical factor, for curvatures of such tissues may render the access thereto more or less difficult and a degree of exposure of the spot through such tissues may also determine ease of access thereto. In another example, the quotient may connote a time required for a subject to reach an orgasm under a preset sexual stimuli, an energy to be spent for the subject to reach the orgasm, a number of orgasms the subject may reach during a single intercourse, and the like. When desirable, the quotient may be arranged to represent one or more of the configurational characteristics, dynamic patterns, and sensitivities of the pelvic and/or penile structures.

In another aspect of the present invention, pelvic quotient systems may be arranged to provide users with emblems which are laden with various variables representing physiologic states of pelvic and/or penile structures and expressing vaginal and/or penile quotients thereby. FIGS. 3A to 3L show schematic views of exemplary emblems carrying various quotients for pelvic and/or penile structures according to the present invention. It is to be understood that various embodiments of such an aspect of this invention may be deemed as a three-dimensional expansion of those formats expressing such quotients as patterns and/or images defining preset gray-scales or colors. Accordingly, details of the above patterns, images, and/or colors may also apply to various embodiments to be set forth below. In addition, other formats may be implemented into the following embodiments. It is further appreciated that such emblems may be provided only for females or only for males. In order to compare and/or to match such emblems, however, such emblems are provided for both females and males in the same or similar configurations and/or formats. It is appreciated in FIGS. 3A through 3L that emblems on the left side and in the center correspond to those male and female emblems and that emblems on the right side correspond to such emblems disposed close to each other or one over the other for comparing a degree of matching between such emblems and, therefore, a degree of matching between the female and male quotients.

In one exemplary embodiment of this aspect of the present invention and as described in FIG. 3A, the vaginal and penile quotients may be expressed into exemplary emblems provided in a pair of a male emblem 7M and a female emblem 7F. Each emblem 7M, 7F may have a preset shape and/or size and may optionally be assigned with a gray-scale or color, in which such a shape, size, and/or color may be arranged to express each quotient. Such emblems 7M, 7F are to be overlapped one over the other, and the degree of matching therebetween may be assessed primarily based on the matching of their shapes, sizes, orientations, and/or colors, as described hereinabove. Because the emblems 7M, 7F allow comparison of a single value or variable therebetween, these emblems 7M, 7F may best suit those quotients provided in the format of a single number or symbol. When desirable, one or both of such emblems 7M, 7F may further define multiple sections therein either vertically or horizontally such that overlapping one emblem over the other may allow comparison of other states, thereby assessing the matching between the quotients more accurately. Such emblems 7M, 7F may also be arranged to represent other values or variables in different orientations such that rotation of one of such emblems 7M, 7F and/or flipping of one of such emblems 7M, 7F may also allow comparison of multiple values or variables for better assessment of quotient matching.

In another exemplary embodiment of such an aspect of the present invention and as described in FIG. 3B, the vaginal and penile quotients may be expressed into exemplary emblems also provided in a pair of a male emblem 7M and a female emblem 7F. Each emblem 7M, 7F may define a preset shape and/or size and may optionally be assigned with a gray-scale or color, where the shape, size, and/or color may be arranged to express each quotient. In contrary to those shown in FIG. 3A disposed one over the other for comparison, the emblems 7M, 7F of FIG. 3B are to be coupled to each other side by side so that the degree of matching therebetween may be assessed primarily based on the matching of their shapes, sizes, orientations, and/or colors along adjoining edges. Because multiple traits may be embedded along such edges, the emblems 7M, 7F allow comparison of multiple values or variables therebetween. When desirable, multiple edges of such emblems may be arranged to represent such values or variables to allow further comparison of other states of such structures. In addition, such emblems 7M, 7F may also be arranged to represent other values or variables in different orientations such that rotation of one of such emblems 7M, 7F and/or flipping of one of such emblems 7M, 7F may also allow comparison of multiple values or variables for better assessment of quotient matching.

In another exemplary embodiment of such an aspect of the present invention and as described in FIG. 3C, the vaginal and penile quotients may be expressed into exemplary emblems also provided in a pair of a male emblem 7M and a female emblem 7F. Each emblem 7M, 7F may define a preset shape and/or size and may optionally be assigned with a gray-scale or color, where the shape, size, and/or color may be arranged to express each quotient. Similar to those of FIG. 3A, the emblems 7M, 7F may be overlapped one over the other so as to assess the degree of matching therebetween. However, because the male emblem 7M defines at least one protrusion 7P and the female emblem 7F defines a matching indentation 71, the emblems 7M, 7F may be overlapped onto each other while disposing the protrusion 7P inside the indentation 71. Depending upon orientations of such protrusion 7P as well as indentation 71, the emblems 7M, 7F may have to be overlapped onto each other at a preset angle, and their degree of matching may be assessed primarily based upon the matching of their shapes, sizes, orientations, and/or colors, as described hereinabove. Contrary to those of FIG. 3A, these emblems 7M, 7F may allow comparison of multiple values or variables therebetween. When desirable, one or both of such emblems 7M, 7F may also define multiple sections therein either vertically or horizontally such that overlapping one emblem over the other may allow comparison of other values or variables, thereby assessing the matching between the quotients more accurately. Other configurational and/or operational characteristics of the emblems 7M, 7F of FIG. 3C are similar or identical to those of FIGS. 3A and 3B.

In another exemplary embodiment of such an aspect of the present invention and as described in FIG. 3D, the vaginal and penile quotients may be expressed into exemplary emblems also provided in a pair of a male emblem 7M and a female emblem 7F which are similar or identical to those of FIG. 3A. Each emblem 7M, 7F, however, includes a mark 7S which may be painted over an exterior thereof or embedded therein. Such marks 7S are also arranged that, when the emblems 7M, 7F are overlapped onto each other, the degree of matching between the quotients may be assessed primarily based on the matching between shapes of such marks 7S, their sizes, their orientations, and/or their colors, as described hereinabove. Alternatively, such a degree may be assessed whether such marks 7S may complement each other and form a preset shape when their emblems 7M, 7F are overlapped one over the other. Such emblems 7M, 7F may allow comparison of a single value or variable or, alternatively, may include multiple marks 7S for multiple values or variables. When desirable, one or both of such emblems 7M, 7F may further define multiple sections with the marks 7S either vertically or horizontally such that overlapping one emblem over the other may allow comparison of other states. The emblems 7M, 7F may also be arranged to represent other values or variables in different orientations such that rotation of one of such emblems 7M, 7F and/or flipping of one of such emblems 7M, 7F may also allow comparison of multiple values or variables for better assessing the matching between such quotients. Other configurational and/or operational characteristics of the emblems 7M, 7F of FIG. 3D are similar or identical to those of FIGS. 3A to 3C.

In another exemplary embodiment of such an aspect of the present invention and as described in FIG. 3E, the vaginal and penile quotients may be expressed into exemplary emblems also provided in a pair of a male emblem 7M and a female emblem 7F which are similar to those of FIG. 3B. However, such emblems 7M, 7F may further define along coupling edges a preset number of protrusions and/or indentations and/or a preset curvature which may be arranged to express the quotients. Accordingly, the degree of matching between the quotients may be assessed by matching the coupling edges side by side and by comparing the matching of their shapes, sizes, orientations, and/or colors along such edges. For example, a perfect fit along the edges may be deemed as a perfect match between such quotients, while a degree of mismatch may be deemed to be proportional to a size and/or a number of gaps formed along such edges. Because numerous values or variables may be imparted along such edges, such emblems 7M, 7F may also be suitable to compare multiple traits of the vaginal and penile quotients. When desirable, multiple edges of at least one of such emblems may define the curvature, protrusions, and/or indentations in order to represent more values or variables related to the quotients. In addition, such emblems 7M, 7F may also represent other values or variables in different orientations such that flipping of one of such emblems 7M, 7F may allow comparison of multiple values or variables for better assessment of quotient matching. It is appreciated that such protrusions and/or indentations of both of the emblems 7M, 7F generally extend perpendicular to surfaces thereof. In the alternative, such protrusions and/or indentations may extend at preset angles to such surfaces, thereby allowing more values or variables to be expressed along such edges. Other configurational and/or operational characteristics of the emblems 7M, 7F of FIG. 3E are similar or identical to those of FIGS. 3A to 3D.

In another exemplary embodiment of such an aspect of the present invention and as described in FIG. 3F, the vaginal and penile quotients may be expressed into exemplary emblems also provided in a pair of a male emblem 7M and a female emblem 7F which amount to a variation of those of FIGS. 3A and 3E and/or a variation of that of FIG. 3C. More specifically, each emblem 7M, 7F may form multiple protrusions 7P and/or indentations 71 extending from one edge to an opposing edge. Accordingly, the degree of matching between the quotients may be assessed by overlapping such coupling surfaces one over the other and comparing the matching of their shapes, sizes, orientations, and/or colors over such surfaces. For example, a perfect fit between the surfaces may be deemed as a perfect match between such quotients, while a degree of mismatch may be deemed to be proportional to a space or a gap formed along the surfaces. Because numerous values or variables may be imparted over such surfaces, the emblems 7M, 7F may also be suitable to compare multiple traits of the vaginal and penile quotients. When desirable, one or both of such emblems 7M, 7F may define multiple sections therein either vertically or horizontally such that overlapping one emblem over the other may allow comparison of other values or variables, thereby assessing the matching between the quotients more accurately. In addition, such emblems 7M, 7F may also represent other values or variables in different orientations such that rotating of one of the emblems 7M, 7F may allow comparison of multiple values or variables for better assessment of quotient matching. It is appreciated that such protrusions and/or indentations of both of the emblems 7M, 7F generally extend parallel to each other. Alternatively, such protrusions and/or indentations may instead extend at preset angles with respect to each other, thereby allowing more values or variables to be expressed along such edges. Other configurational and/or operational characteristics of the emblems 7M, 7F of FIG. 3F are similar or identical to those of FIGS. 3A to 3E.

In another exemplary embodiment of such an aspect of the present invention and as described in FIG. 3G, the vaginal and penile quotients may be expressed into exemplary emblems also provided in a pair of a male emblem 7M and a female emblem 7F which are generally similar to those of FIG. 3E. However, the female emblem 7F defines an opening 70 therein, while the male emblem 7M is shaped and sized to be disposed inside the opening 70. In addition, such emblems 7M, 7F may form a preset number of protrusions and indentations or may form a preset curvature around their circumferences which may express the quotients. Therefore, the degree of matching between the quotients may be assessed by inserting the male emblem 7M into the opening 70 of the female emblem 7F, by matching the circumferences of such emblems 7M, 7F, and by comparing such matching of their shapes, sizes, orientations, and/or colors along the edges. For example, a perfect fit along the circumferences may be deemed as a perfect match between such quotients, while a degree of mismatch may be deemed to be proportional to a size and/or a number of gaps formed along such edges. Because numerous values or variables may be imparted along such edges, such emblems 7M, 7F may also be suitable to compare multiple traits of the vaginal and penile quotients. When desirable, curvatures of both of the emblems 7M, 7F may be formed in various directions which may be parallel to, perpendicular to, or at preset angles to surfaces of the emblem 7M, 7F, thereby allowing more values and/or variables to be expressed along such edges. Other configurational and/or operational characteristics of the emblems 7M, 7F of FIG. 3G are similar or identical to those of FIGS. 3A to 3F.

In another exemplary embodiment of such an aspect of the present invention and as described in FIG. 3H, the vaginal and penile quotients may be expressed into exemplary emblems also provided in a pair of a male emblem 7M and a female emblem 7F both of which are typically similar to those of FIG. 3G. However, the male emblem 7M is preferably shaped and sized to perfectly fit into the opening 70 of the female emblem 7F. In addition, the female emblem 7F may also be provided with various marks 7S around the opening 70, while the male emblem 7M may similarly be provided with various marks 7S around its periphery. Therefore, the degree of matching between the quotients may be assessed by fitting the male emblem 7M into the opening 70 of the female emblem 7F, by disposing the male emblem 7M in a preset orientation or rotating the male emblem 7M into a desirable orientation, and by counting a number of marks 7S of the female emblem 7F matching those of the male emblem 7M. For example, a perfect matching between all marks 7S may be deemed as a perfect match between such quotients, and a number of mismatched marks 7S may be deemed to be proportional to a mismatch between the quotients. Because numerous values or variables may be imparted along such an opening 70, such emblems 7M, 7F may be suitable to compare multiple traits of the vaginal and penile quotients. When desirable, such marks 7S may be provided in multiple concentric rows, may also be provided on both surfaces of the emblems 7M, 7F, thereby allowing more values or variables to be expressed on such emblems 7M, 7F. Further configurational and/or operational characteristics of the emblems 7M, 7F of FIG. 3H are similar or identical to those of FIGS. 3A to 3G.

In another exemplary embodiment of such an aspect of the present invention and as described in FIG. 3I, the vaginal and penile quotients may be expressed into exemplary emblems also provided in a pair of a male emblem 7M and a female emblem 7F. Each emblem 7M, 7F may define a preset shape and/or size and may optionally be assigned with a gray-scale or color, where the shape, size, and/or color may be arranged to express each quotient. The emblems 7M, 7F are further arranged to couple with each other side by side and to form an object which may define a preset shape such as a star in this embodiment. Accordingly, the degree of matching between such quotients may be assessed by coupling the emblems 7M, 7S side by side along their coupling edges and by analyzing how close the emblems 7M, 7F may approximate the object with the known shape. In the alternative, such emblems 7M, 7F may be arranged to always form the exact shape of the object. However, the colors or marks provided on such emblems 7M, 7F may be used to assess the degree of matching of such quotients. The coupling edges of such emblems 7M, 7F may be imparted with many values or variables such that various characteristics of the pelvic and penile structures may be matched thereby. When desirable, one or both of such emblems 7M, 7F may form multiple sections therein either vertically or horizontally so that coupling such emblems 7M, 7F may allow comparison of other states. Further configurational and/or operational characteristics of the emblems 7M, 7F of FIG. 3I may be similar or identical to those of FIGS. 3A to 3H.

In another exemplary embodiment of such an aspect of the present invention and as described in FIG. 3J, the vaginal and penile quotients may be expressed into exemplary emblems also provided in a pair of a male emblem 7M and a female emblem 7F which may be similar to those of FIGS. 3G and 3H. However, the female emblem 7F defines an opening 70 therein and includes a cover 3C, and the male emblem 7M is shaped and sized to be releasably disposed inside the opening 70 of the female emblem 7F. Accordingly, the degree of matching between such quotients may be assessed by disposing the male emblem 7M inside the opening 70 of the female opening 7F, by closing the cover 7C and retaining the male emblem 7M inside the female emblem 7F, and by analyzing how close the emblems 7M, 7F fit each other. For example, shaking the female emblem 7F with its cover 7C closed and analyzing any sound generated thereby may manifest how close the male emblem 7M may fit the opening 70 of the female emblem 7F. In the alternative, such emblems 7M, 7F may be arranged to always from the exact shape of the object. However, the colors or marks provided on such emblems 7M, 7F may be used to assess the degree of matching of such quotients. In addition, the coupling edges of the emblems 7M, 7F may be imparted with many values or variables such that various characteristics of the pelvic and penile structures may be matched thereby. When desirable, one or both of such emblems 7M, 7F may form multiple sections therein either vertically or horizontally such that coupling such emblems 7M, 7F may allow comparison of other states. Other configurational and/or operational characteristics of the emblems 7M, 7F of FIG. 3J are similar or identical to those of FIGS. 3A to 3I.

In another exemplary embodiment of such an aspect of the present invention and as described in FIG. 3K, the vaginal and penile quotients may be expressed into exemplary emblems also provided in a pair of a male emblem 7M and a female emblem 7F which may be typically similar to those of FIGS. 3E to 3G but which may be deemed as a three-dimensional expansion thereof. For example, the female emblem 7F forms an opening defining a continuous indentation 71 defining a curvature along an axis of the emblem 7F, while the male emblem 7M defines a continuous protrusion 7P extending outward and forming a curvature along an axis of the emblem 7M. Such three-dimensional protrusion 7P as well as indentation 71 may express numerous value or variable of the pelvic and penile structures. Therefore, the degree of matching between such quotients may be assessed by disposing the male emblem 7M inside the opening 70 of the female opening 7F and by analyzing how close the emblems 7M, 7F may fit each other. For example, a perfect matching between the protrusion 7P and indentation 71 may be deemed as a perfect match between such quotients, while a number and/or size of any gap formed therebetween may be deemed to be proportional to a mismatch between the quotients. Alternatively, such emblems 7M, 7F may be arranged to always fit other, while the colors or marks provided on the emblems 7M, 7F may be used to assess the degree of matching of such quotients. When desirable, one or both of such protrusion 7P and indentation 71 may form multiple sections therein either vertically or horizontally such that coupling such emblems 7M, 7F may allow comparison of other states. Other configurational and/or operational characteristics of the emblems 7M, 7F of FIG. 3K may be similar or identical to those of FIGS. 3A to 3J.

In another exemplary embodiment of such an aspect of the present invention and as described in FIG. 3L, the vaginal and penile quotients may be expressed into exemplary emblems also provided in a pair of a male emblem 7M and a female emblem 7F which are similar to those of FIG. 3K. However, the female emblem 7F is arranged to form an opening defining therealong one or more protrusions 7P (or indentations) and to include one or multiple movable marks 7S. The male emblem 7M is shaped and sized to be releasably inserted into such an opening 70 of the female emblem 7F and similarly defines therealong one or more indentations 71 (or protrusions). Such marks 7S of the female emblem 7F may be disposed to be flush with a surface of the emblem 7M in their default positions but to pop up above such a surface as the protrusion 7P of the female emblem 7F may be abutted by the indentation 71 of the female emblem 71. Therefore, the degree of matching between such quotients may be assessed by inserting the male emblem 7M through the opening 70 of the female opening 7F and by finding how many pop-up marks 7S of the female emblem 7F may pop up by such a male emblem 7M. For example, when all of such marks 7S pop up from their default positions, such female and male quotients may be deemed to perfectly match each other, whereas a number of such marks 7S remaining in their default positions may be deemed to be proportional to a mismatch between such quotients. When desirable, one or both of such protrusion 7P and indentation 71 may form multiple sections therein either vertically or horizontally such that coupling such emblems 7M, 7F may allow comparison of other states. Other configurational and/or operational characteristics of the emblems 7M, 7F of FIG. 3L may also be similar or identical to those of FIGS. 3A to 3K.

Configurational and/or operational variations and/or modifications of the above embodiments of the exemplary systems and various members thereof described in FIGS. 3A to 3L may also fall within the scope of this invention.

The above female and male emblems may have any arbitrary shapes and/or sizes as long as such emblems may be overlapped onto and/or coupled to each other in order to assess the match or mismatch therebetween. As described above, various values and/or variables of the pelvic and/or penile structures may be expressed on such emblems in many arrangements such as, e.g., shapes or sizes of such emblems, colors thereof, curvatures thereof, surface textures thereof, and the like. As long as they may be readily identified by the users, almost any configurational characteristics may be employed to express various values or variables of such structures.

It is appreciated that any of the above embodiments for various emblems may be dimensionally expanded or reduced. In general, dimensional expansion may allow more values and variables to be expressed by such emblems, whereas dimensional reduction may allow less values and variables to be expressed thereby. The dimensional expansion may also be performed for various reasons. For example, the expansion may be preferred to increase the size of the emblem for better handling such. In another example, the expansion may be performed to increase an amount of traits to be expressed thereby. In either of these cases, such expansion may be performed symmetrically, i.e., the shape of the emblem may be maintained along a direction of expansion. Alternatively, such expansion may be performed asymmetrically such that more values or variables may be imparted to the shapes of such an emblem along the direction of expansion.

Any of the above emblems may be arranged to consist of multiple segments. Therefore, more values or variables may be represented along coupling edges of the emblems. In the alternative, such modular configurations may allow the users to match different segments of the emblems in different arrangements.

It is appreciated that the above emblems may incorporate various features of other emblems of other embodiments. In addition, the emblems may incorporate other formats of expressing the values or variables as described above. Accordingly, such color format, image format, and/or sound format may also be incorporated into the emblems of FIGS. 3A to 3L.

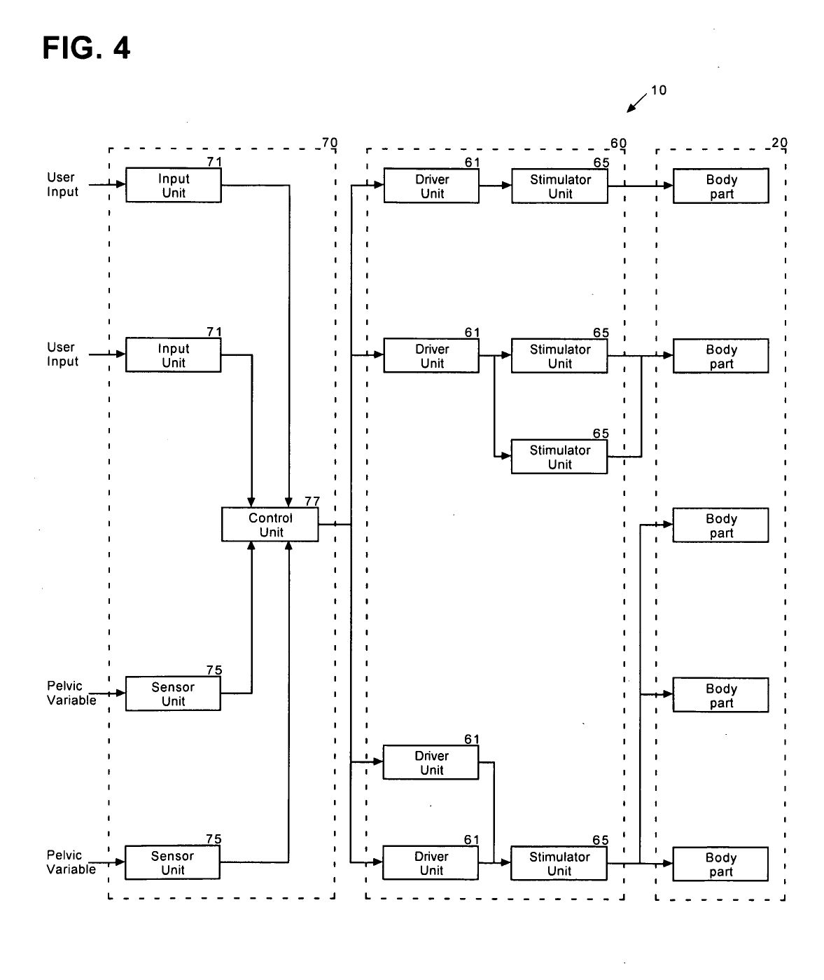

In another aspect of the present invention, an exemplary pelvic quotient system may include at least one body member, at least one (optional) actuator member, at least one control member, at least one power (supply) member, and the like. FIG. 4 describes a schematic diagram of various functional members and units of an exemplary pelvic quotient system according to the present invention. Such a pelvic quotient system 10 may include at least one body member 20, at least one actuator member 60, at least one control member 70, and at least one power (supply) member (not included in this figure) which may be a wire and plug assembly for receiving AC power from an electric outlet or may instead be a dry-cell battery or a rechargeable battery.

As briefly described in FIGS. 1A to 2D, the body member 20 includes at least one first unit and at least one second unit, where the first unit defines various parts capable of contacting one or more portions of the pelvic structure when engaging therewith and optionally capable of providing various stimuli through one or more movements thereof. The body parts defining various configurations may be generally similar to those of the prior art devices, whereas various novel body parts are provided in the co-pending Applications.

The actuator member 60 includes at least one driver unit 61 and at least one stimulator unit 65. The main function of the actuator member 60 is to effect one or multiple movements of one or multiple parts of the body member 20 (i.e., the body parts of the first unit thereof). To this end, the driver unit 61 receives electric power from the power member and generates driving force which is transferred to the stimulator unit 65 through at least one power transmission unit (not included in this figure). The driving unit 61 may therefore be arranged to supply the air, gas, and/or fluid into the inflatable chamber and/or discharge such out of the chamber for the baseline adjustment as disclosed in the co-pending Applications. The stimulator unit 65 may not be necessary for various pelvic quotient systems of this invention, but may be arranged to receive the driving force, to optionally convert the driving force into actuating force, and then to effect desirable movement of a preset part of the first unit when such a system may be utilized as various pelvic relaxing systems described in the co-pending Applications. The stimulator unit 65 may manipulate the driver unit 61 and change the configuration of the first unit 21. Other details of this optional actuator member have been provided in the co-pending Applications.

The control member 70 may include at least one control unit 77 and at least one of at least one input unit 71 and sensor unit 75. The main function of the control member 70 is to generate signals for measuring various variables representing the physiological states of the pelvic structure. To this end, such a sensor unit 75 may be arranged to measure various pelvic variables and to generate sensing signals in response thereto, while the control unit 77 may be arranged to process the sensing signals, to obtain or assess the pelvic variables therefrom, and to provide values of the variables to the user. As disclosed in the co-pending Applications, the input unit 71 and sensor unit 75 may generally refer to similar articles capable of receiving various user inputs and generating various signals in response thereto. Within the scope of the present invention, those articles incorporated into the first unit are to be referred to as the sensor units 75, while those incorporated into the second unit are referred to as the input units 71, unless otherwise specified. Thus, the input unit 71 of the pelvic quotient system 10 generally stays outside the pelvic structure and receives the user input through her hand, whereas the sensor unit 75 of the pelvic quotient system 10 is preferably disposed on, over or inside the pelvic structure and receives various pelvic variables through various portions of the pelvic structure. Other configurational details of the input unit 71 of the control member 60 have already been disclosed in the co-pending Applications and will be omitted for ease of illustration.

Another main function of the control member 70 is to convert the values of various pelvic and penile variables into the vaginal the penile quotients, respectively. To this end, the sensor unit 75 may be arranged to measure various pelvic variables and to generate sensing signals in response thereto, while the control unit 77 may be arranged to process the sensing signals, to assess the pelvic and/or penile variables therefrom, and to convert such values into such quotients based on a preset format. When the pelvic or penile quotients may be assessed in various formats, the control unit 77 may also receive a control signal from the user and assess such quotients accordingly. In the alternative, such a control unit 77 may provide a selection to the user and assess the quotients according thereto. The control unit 77 may also be arranged to assess such a degree of matching between the vaginal and penile quotients based on various formats as described above.

Although not shown in the figure, the control member 70 may include at least one audio and/or visual input unit, at least one audio and/or visual output unit, at least one storage unit, and at least one processing unit. The control member 70 may also include other units such as, e.g., at least one driver unit, one or more signal paths between various members and/or units of such a system 10, and so on.

The storage unit may store various audio and/or video signals temporarily or permanently. The storage unit may be any conventional data storage articles such as, e.g., magnetic tapes and/or disks, optical disks, semiconductor chips, and other data storage devices capable of storing analog and/or digital data therein. Depending on types of the devices, the control member may also include suitable drivers to operate the storage unit, where examples of such drivers may include, but not be limited to, magnetic tape or disk drivers, optical tape or disk drivers, circuits for locating and retrieving desired signals, and the like. Such signals may be classified in various modes, e.g., based upon contents of such signals, their classifications, presence or absence of action bases therein, source thereof, their voice bases, and the like. Therefore, the control member 70 may readily find and retrieve the desired signal from the storage unit. The storage unit may be disposed in various locations of the system 10 and may be exposed or hidden in the first and/or second units. When desirable, the storage unit may be provided as a replaceable cartridge so that the user may load a desired storage unit, change such a unit when used to its full capacity, and the like. The control member 70 may optionally be arranged to communicate with external storage devices in order to send, store, search, and/or retrieve desired signals thereto or therefrom. As will be described in detail below, the storage unit may store various sounds and/or images for expressing the pelvic variables.

It is appreciated within the scope of this invention that any audiovisual signal may be deemed to define at least one of a content basis, a voice basis, an action basis, and a background basis, all of which have been described in the co-pending Applications. With such definitions, the processing unit receives the audiovisual signals and modifies the audio signals by changing, e.g., at least on temporal pattern thereof, their amplitudes, their frequencies, their orders, and the like. The processing unit may also generate compound signals by combining multiple audiovisual signals or, alternatively, generate a synthesized signal by changing and/or replacing at least one basis of such signals, and the like. The processing unit may receive the sensing signals from the sensor unit 75 and assess various variables therefrom as described above.

Through such audiovisual output unit and processing unit, the control member 70 may provide various audiovisual feedback signals to the user according to various modes. In one example, such a control member 70 may provide only audible signals to the user, where such signals may not include any content basis, where such signals may carry one or more content bases related to the averaged or instantaneous absolute value of the variable, the relative value of the variable compared to various preset values such as a preset reference, the user's or other's previous performances in terms of the same or different variables, and the like. The audible signal may instead carry verbal instruction about the pelvic exercise, where such instruction may be prerecorded or provided adaptively in response to the user's performance. In the alternative, such audible signals may be arranged to lead or guide the user during the pelvic practice. Such audible feedback signals are also preferably synchronized with the measured pelvic variables in order to maximize such feedback effect. In another example, such a control member 70 may provide only visual signals to the user, where such signals may be mono- or multichromic signals and where such signals may have a constant intensity or intensities thereof may vary according to various dynamic patterns of the pelvic variables. Such visual signals may similarly be arranged to provide the feedback effect to the user or, conversely, to lead or guide the user during the pelvic exercise. The visual feedback signals are also preferably synchronized with the measured pelvic variables to maximize the feedback effect. In another example, the control member 70 may also provide only tactile signals to the user, where such signals may be directed to the pelvic structure of the user, to other body parts of the user, and/or to a third party who may be assisting the user during the pelvic exercise. The tactile signals may define uniform intensities and may be applied to the same portion of the user. Alternatively, such signals may define different intensities and may be applied to the same or different portions of the user. The tactile signals may similarly be arranged to provide the feedback effect to the user or, conversely, to lead or guide the user during the pelvic exercise. Such tactile feedback signals are also preferably synchronized with the measured pelvic variables so as to maximize the feedback effect.

The control member 70 may display an image of the sensor unit 75 and then superpose such an image by the absolute and/or relative values of the measured variables, with or without displaying locations of the sensor unit 75 measuring such variables. The control member 70 may also display a static and/or dynamic image of an anatomy of the pelvic structure and identify the location of the first unit and/or sensor unit 75 in such an anatomy. This embodiment is particularly useful when the user may change her posture during the exercise and monitor changes in the measured pelvic variables in response to such changes in the postures. The user may also monitor the changes in the measured variables as she may move her leg or thigh, may bend or rotate her back, and so on. In addition, such an embodiment may visually guide the users as to the matching between the quotients, where most of the match or mismatch occurs, and the like.

The control member 70 and its various units may also be arranged to perform other functions so that the pelvic quotient system of this invention may also serve as various pelvic relaxing systems of the co-pending Applications. In this embodiment, various members and units of the pelvic quotient system may be tailored similar to the corresponding members and units of the pelvic relaxing systems as disclosed in the co-pending Applications.

The pelvic quotient system of this invention may be provided as an unitary article. In such an embodiment, the body member of the system may incorporate therein various other members and units of the system. Alternatively, the system may include the body member and the control member which may be detached from the body member and operatively couple with the body member wirelessly or through wire. When desirable, the system may be arranged to utilize external audiovisual devices for playing various sounds and/or displaying various images. Such a system may operatively couple with such external devices through wire or wirelessly. In addition, such a system may be arranged to only include the control member which may then imports various signals related to various pelvic or penile variables. The system may assess therefrom the vaginal or penile quotient and may also assess the match or mismatch between such quotients.

The pelvic quotient system of the present invention may further be construct to be waterproof. For example, various input units of the body member may be covered by a waterproof layer or may be disposed inside the second unit of the body member. In addition, the system may be arranged to run by a rechargeable battery which may be recharged by electromagnetic induction from outside.

Unless otherwise specified, various features of one embodiment of one aspect of the present invention may apply interchangeably to other embodiments of the same aspect of this invention and/or embodiments of one or more of other aspects of the present invention. Therefore, such a sensor unit of one pelvic quotient system of FIGS. 1A to 4D may be replaced interchangeably by the sensor unit of another system thereof. Similarly, various emblems of FIGS. 3A to 3L may be incorporated into those systems of FIGS. 1A to 2D, and the like.

The pelvic quotient system of the present invention may be used in conjunction with various audiovisual aids. For example, the user may play a source of audio signals which generates various audible sounds to which the user may synchronize her pelvic exercise, based upon which the user may perform the exercise, and the like. The control member 70 may be arranged to receive the audio signals, to compare such audio signals with the sensing signals generated by the sensor units, and to provide the user with such feedback audiovisual signals. In another example, the user may turn on a source of visual signals generating various visual signals to which such an user may synchronize the exercise, based upon which the user may perform the exercise, and the like. The control member 70 may be arranged to receive the visual signals, to compare the visual signals with the sensing signals generated by the sensor units, and then to provide the user with the feedback audiovisual signals. In yet another example, the user may play another visual signals generating various images to which the user may synchronize the pelvic exercise, based upon which the user may perform the exercise, and the like. The control member 70 may be arranged to receive the visual signals, to compare the visual signals with the sensing signals generated by the sensor units, and then to provide the user with the feedback audiovisual signals. When desirable, such images may also include conventional exercise routines such as, e.g., yoga, running, body building, and so on. In addition, such audiovisual aids may also be tailored to improve the match between the specific pelvic and penile structure by capitalizing on such vaginal and penile quotients.

It is to be understood that, while various aspects and embodiments of the present invention have been described in conjunction with the detailed description thereof, the foregoing description is intended to illustrate and not to limit the scope of the invention, which is defined by the scope of the appended claims. Other embodiments, aspects, advantages, and modifications are within the scope of the following claims.

What is claimed is:

1. A system capable of assessing at least one quotient which is a set of a plurality of values and represents physiologic states of a pelvic structure including an entry and a wall, wherein said entry is configured to define therethrough an orifice and wherein said wall is configured to have muscles and to define an internal cavity which extends inwardly and which is bound by said muscles, said system comprising:
at least one body member which is configured to include at least one first unit for contacting at least a portion of said pelvic structure when said body member is engaged with said structure; and
at least one control member which is configured to include at least one sensor unit and at least one control unit, wherein said sensor unit is configured to be coupled to said first unit and to measure at least one variable representing said state and wherein said control unit is configured to operatively couple with said sensor unit and to assess said quotient as said set with at least two of said values.

2. The system of claim 1, wherein said variable is at least one of a normal force exerted by said portion of said structure, a bending force exerted thereby, an axial force exerted thereby so as to one of pull and push one of an object and said first unit out of said internal cavity, torque exerted thereby, and a contact between said object and said portion of said structure.

3. The system of claim 1, wherein said variable is at least one of a normal force applied onto at least a part of said first unit, a bending force applied onto said part, an axial force in order to one of pull and push one of said part and an object out of said internal cavity, a torque applied around said part, a velocity of said part, an acceleration thereof, a displacement thereof, a contact between said part and portion of said structure, a dimension of said portion, one of contraction and relaxation of said portion, a duration of at least one of said variables, and a frequency thereof.

4. The system of claim 1, wherein said variable is at least one of a normal force applied onto said portion of said structure, a bending force applied onto said portion, an axial force resisting movement of one of an object and said first unit out of said cavity of said structure, a torque applied about said portion, a velocity of said portion, an acceleration thereof, a displacement thereof, a contact between said portion and at least one part of said first unit, contraction and relaxation of said portion, a duration of at least one of said variables, and a frequency of at least one of said variables.

5. The system of claim 1, wherein said values are configured to relate to a single variable and to be measured by said sensor unit in a plurality of different portions of said structure.

6. The system of claim 1, wherein said values are configured to relate to a single variable and to be measured by said sensor unit in a plurality of different timings in said structure.

7. The system of claim 1, wherein said values are configured to relate to a plurality of different variables and to be measured by said sensor unit in said portion of said structure.

8. The system of claim 7, wherein at least one of said values is configured to relate to a dynamic pattern of movement of said structure and wherein at least another of said values is configured to be related to a configuration of said structure.

9. The system of claim 8, wherein said dynamic pattern includes at least one of

10. The system of claim 8, wherein said configuration includes at least one of

11. The system of claim 1, wherein said sensor unit measures said values while an user maintains said structure in a preset posture.

12. The system of claim 1, wherein said sensor unit measures said values while an user changes postures of said structure.

13. The system of claim 12, wherein said user changes said postures by moving at least one of her leg, thigh, back, and abdomen.

14. The system of claim 1, wherein said sensor unit is configured to operate calibrated on a preset baseline, wherein at least one of said values is obtained without adjusting said baseline, and wherein at least another of said values is obtained after adjusting said baseline.

15. The system of claim 1, wherein said control member is also configured to include at least one audiovisual output unit and to generate thereby at least one of sounds and images each determined by said values.

16. The system of claim 1 further comprising at least one emblem which is configured to be one of a two-dimensional object and a three-dimensional object and to define at least one of a shape, a size, and a color each of which is determined by said values.

17. A system capable of assessing a plurality of quotients each describing physiologic states of a pelvic structure including an entry and a wall, wherein said entry is configured to define therethrough an orifice and wherein said wall is configured to include muscles and to form an internal cavity which extends inwardly and which is bound by said muscles, said system comprising:
at least one body member which is configured to include at least one first unit for contacting at least a portion of said pelvic structure when said body member is engaged with said structure; and
at least one control member which is configured to include at least one sensor unit and at least one control unit, wherein said sensor unit is configured to be coupled to said first part and to measure at least one variable representing said state and wherein said control unit is configured to operatively couple with said sensor unit, to obtain a value of said variable in each of a plurality of postures of said structure, to assess each of said quotients from said value in each of said postures, and to provide to an user said quotients and said postures corresponding thereto.

18. The system of claim 17, wherein said control unit is also configured to provide an user with a plurality of said postures in each of which said quotient is configured to exceed a preset threshold.

19. A method of assessing at least one quotient which represents a physiologic state of a pelvic structure with an entry and a wall, wherein said entry is configured to define therethrough an orifice and wherein said wall is configured to include muscles and to form an internal cavity which extends inwardly and which is bound by said muscles, said method comprising the steps of:
contacting at least a portion of said pelvic structure;
measuring a plurality of variables each representing different characteristics of said state in a plurality of portions of said structure one at a time;
obtaining a plurality of values of said variable by said measuring; and
assessing said quotient as a set including at least two of said values of said portions.

20. The method of claim 19 further comprising the steps of:
obtaining at least one mathematical equation as a function of at least one of said variables;
assessing at least another value by plugging at least one of said values into said equation; and
including said assessed value in said set of said values.
